(19) United States
(12) Patent Application Publication
(10) Pub. No.: US 2024/0126169 A1
(43) Pub. Date: Apr. 18, 2024

(54) PHOTOSENSITIVE RESIN COMPOSITION AND DISPLAY DEVICE

(71) Applicants: Samsung Display Co., Ltd., Yongin-si (KR); DUK SAN NEOLUX CO., LTD., Cheonan-si (KR)

(72) Inventors: YANG-HO JUNG, Yongin-si (KR); BUM SUNG LEE, Cheonan-si (KR); CHANG MIN LEE, Cheonan-si (KR); JUN BAE, Cheonan-si (KR); NAKCHO CHOI, Yongin-si (KR); HOON KANG, Yongin-si (KR); DAE-GI KWEON, Yongin-si (KR); JUNGI KIM, Yongin-si (KR); JIHEE KIM, Yongin-si (KR); JUNHO SIM, Yongin-si (KR); JAEHUN LEE, Yongin-si (KR)

(21) Appl. No.: 18/373,936

(22) Filed: Sep. 27, 2023

(30) Foreign Application Priority Data
Sep. 28, 2022 (KR) 10-2022-0123777

(51) Int. Cl. G03F 7/031; C07C 57/02; G03F 7/038; H10K 59/122

(52) U.S. Cl. CPC G03F 7/031; C07C 57/02; G03F 7/038; H10K 59/122

(57) ABSTRACT

Provided is a photosensitive resin composition including a reactive unsaturated compound, an alkali soluble resin, an initiator, and a pigment. The reactive unsaturated compound is selected to cause the composition to exhibit high-resolution pattern properties that facilitate forming a pixel defining film in a display device. The reactive unsaturated compound may be an acryl-based compound having at least three ethylenically unsaturated double bond groups in molecules and having a viscosity of about 200 mPa s to about 280 mPa s at 40° C. A display device including such pixel defining film is also presented.

### CROSS-REFERENCE TO RELATED APPLICATIONS

This U.S. non-provisional patent application claims priority under 35 U.S.C. § 119 of Korean Patent Application No. 10-2022-0123777 filed on Sep. 28, 2022, the entire contents of which are hereby incorporated by reference.

### BACKGROUND

The present disclosure relates to a photosensitive resin composition and a display device, and more particularly, to a display device including a photosensitive resin composition and a pixel defining film formed using the photosensitive composition.

Various types of display devices used for multimedia devices such as a television set, a mobile phone, a tablet computer, a navigation system, and a game console are being developed. When external light is provided to the display devices while in use, the provided external light is reflected from an electrode in a display panel, negatively affecting the display quality.

A pixel defining film formed of a photosensitive resin composition including a pigment may be applied to the display devices to prevent reflection and defects that may cause poor expression of black color.

However, a disadvantage of using a general photosensitive resin composition is that it often causes pattern breakup or residue in a photolithography process.

### SUMMARY

The present disclosure provides a photosensitive resin composition having excellent developability and processability.

The present disclosure also provides a display device including a pixel defining film formed using the photosensitive resin composition.

An embodiment of the inventive concept provides a photosensitive resin composition including a reactive unsaturated compound, an alkali soluble resin, an initiator, and a pigment. The reactive unsaturated compound may include an acryl-based compound having at least three ethylenically unsaturated double bond groups in molecules and having a viscosity of about 200 mPa·s to about 280 mPa·s at 40° C.

In an embodiment, the acryl-based compound may be represented by Formula 1 below.

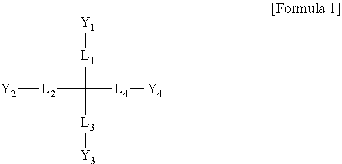

In Formula 1 above, L₁ to L₄ may each independently be a substituted or unsubstituted alkylene group having 1 to 25 carbon atoms, and Y₁ to Y₄ may each independently be a hydrogen atom, a hydroxy group or a substituted or unsubstituted acrylic acid derivative. At least three of Y₁ to Y₄ may include a substituted or unsubstituted acrylic acid derivative.

In an embodiment, the acryl-based compound represented by Formula 1 above may be represented by Formula 2 or Formula 3 below.

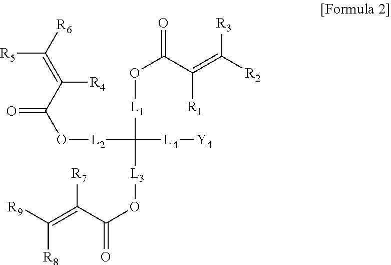

In Formula 2 above, L₁ to L₄ may be the same as defined in Formula 1 above, R₁ to R₉ may each independently be a hydrogen atom, a deuterium atom, a substituted or unsubstituted alkyl group having 1 to 20 carbon atoms, or a substituted or unsubstituted aryl group having 6 to 24 ring-forming carbon atoms, and Y₄ may be a hydrogen atom or a hydroxy group.

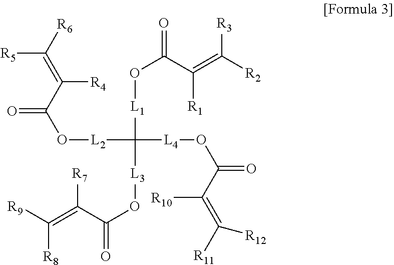

In Formula 3 above, L₁ to L₄ may be the same as defined in Formula 1 above, and R₁ to R₁₂ may each independently be a hydrogen atom, a deuterium atom, a substituted or unsubstituted alkyl group having 1 to 20 carbon atoms, or a substituted or unsubstituted aryl group having 6 to 24 ring-forming carbon atoms.

In an embodiment, the reactive unsaturated compound may further include at least one of a monomer or an oligomer, which has at least one ethylenically unsaturated double bond.

In an embodiment, the alkali soluble resin may include at least one of a cardo-based resin, an acryl-based resin, a polyester-based resin, a polyurethane-based resin, a polysiloxane resin, a polycyclic side chain-containing resin, or an acid-modified epoxy resin. The cardo-based resin may have a weight average molecular weight of about 1,000 g/mol to about 100,000 g/mol.

In an embodiment, the pigment may include at least one of a black organic pigment, a black inorganic pigment, or a mixture of two or more coloring pigments.

In an embodiment, the photosensitive resin composition may further include a solvent.

In an embodiment, with respect to a total weight of a composition, the photosensitive resin composition may include the acryl-based compound in an amount of about 0.3 wt % to about 30 wt %, the alkali soluble resin in an amount of about 1 wt % to about 30 wt %, the initiator in an amount of about 0.01 wt % to about 10 wt %, the pigment in an amount of about 1 wt % to about 30 wt %, and the solvent in a residual amount.

In an embodiment, the black organic pigment may include at least one of a benzofuranone-based black pigment, a perylene-based black pigment, an azo-based black pigment, lactam black, aniline black, or an indolinone-based black pigment. The black organic pigment may include a coating layer on a surface, and the coating layer may include at least one of a silica coating layer, a metal oxide coating layer, or a metal hydroxide coating layer.

In an embodiment of the inventive concept, a display device includes a display panel including a plurality of light emitting elements and a pixel defining film separating the plurality of light emitting elements, and a light control layer disposed on the display panel. The pixel defining film may be formed of the photosensitive resin composition described above.

### BRIEF DESCRIPTION OF THE FIGURES

The accompanying drawings are included to provide a further understanding of the inventive concept, and are incorporated in and constitute a part of this specification. The drawings illustrate embodiments of the inventive concept and, together with the description, serve to explain principles of the inventive concept. In the drawings:

FIG. 1 is an exploded perspective view of a display device according to an embodiment;

FIG. 2 is a cross-sectional view of a display module according to an embodiment;

FIG. 3 is a cross-sectional view of a display panel according to an embodiment;

FIG. 4 is a plan view enlarging and showing a portion of a display region shown in FIG. 1;

FIG. 5 is a cross-sectional view of a display module according to an embodiment, corresponding to line II-IT of FIG. 4;

FIG. 6A is an image showing results of evaluation on resolution and adhesion of Example 1;

FIG. 6B is an image showing results of evaluation on resolution and adhesion of Comparative Example 1;

FIG. 6C is an image showing results of evaluation on resolution and adhesion of Comparative Example 2; and

FIG. 6D is an image showing results of evaluation on resolution and adhesion of Comparative Example 3.

### DETAILED DESCRIPTION

The present disclosure may be modified in many alternate forms, and thus specific embodiments will be exemplified in the drawings and described in detail. It should be understood, however, that it is not intended to limit the present disclosure to the particular forms disclosed, but rather, is intended to cover all modifications, equivalents, and alternatives falling within the spirit and scope of the present disclosure.

As used herein, when an element (or a region, a layer, a portion, etc.) is referred to as being “on,” “connected to,” or “coupled to” another element, it means that the element may be directly disposed on/connected to/coupled to the other element, or that a third element may be disposed therebetween.

Like reference numerals refer to like elements. In addition, in the drawings, the thickness, the ratio, and the dimensions of elements are exaggerated for an effective description of technical contents.

The term “and/or,” includes all combinations of one or more of which associated configurations may define.

It will be understood that, although the terms first, second, etc. may be used herein to describe various elements, these elements should not be limited by these terms. These terms are only used to distinguish one element from another element. For example, a first element may be referred to as a second element, and similarly, a second element may be referred to as a first element without departing from the teachings of the present disclosure. The singular forms are intended to include the plural forms as well, unless the context clearly indicates otherwise.

In addition, terms such as “below,” “lower,” “above,” “upper,” and the like are used to describe the relationship of the configurations shown in the drawings. The terms are used as a relative concept and are described with reference to the direction indicated in the drawings.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It will be further understood that the terms “includes” or “including”, when used in this specification, specify the presence of stated features, integers, steps, operations, elements, components, or a combination thereof, but do not preclude the presence or addition of one or more other features, integers, steps, operations, elements, components, or combinations thereof.

Unless explicitly stated, with regard to the term “a substituted or unsubstituted”, “substituted” indicates substitution with at least one substituent selected from the group consisting of a C₂-C₂₀ heterocyclic group including at least one heteroatom selected from the group consisting of O, N, S, Si, and P, a deuterium atom, a halogen atom, an amino group, a nitrile group, a nitro group, a C₁-C₂₀ alkyl group, a C₁-C₂₀ alkoxy group, a C₁-C₂₀ alkylamine group, a C₁-C₂₀ alkylthiophene group, a C₆-C₂₀ arylthiophene group, a C₂-C₂₀ alkenyl group, a C₂-C₂₀ alkynyl group, a C₃-C₂₀ cycloalkyl group, a C₆-C₂₀ aryl group, a deuterium-substituted C₆-C₂₀ aryl group, a C₅-C₂₀ arylalkenyl group, a silane group, a boron group, and a germanium group, and is not limited to these substituents.

Hereinafter, a photosensitive resin composition and a display device according to an embodiment of the inventive concept will be described with reference to the accompanying drawings.

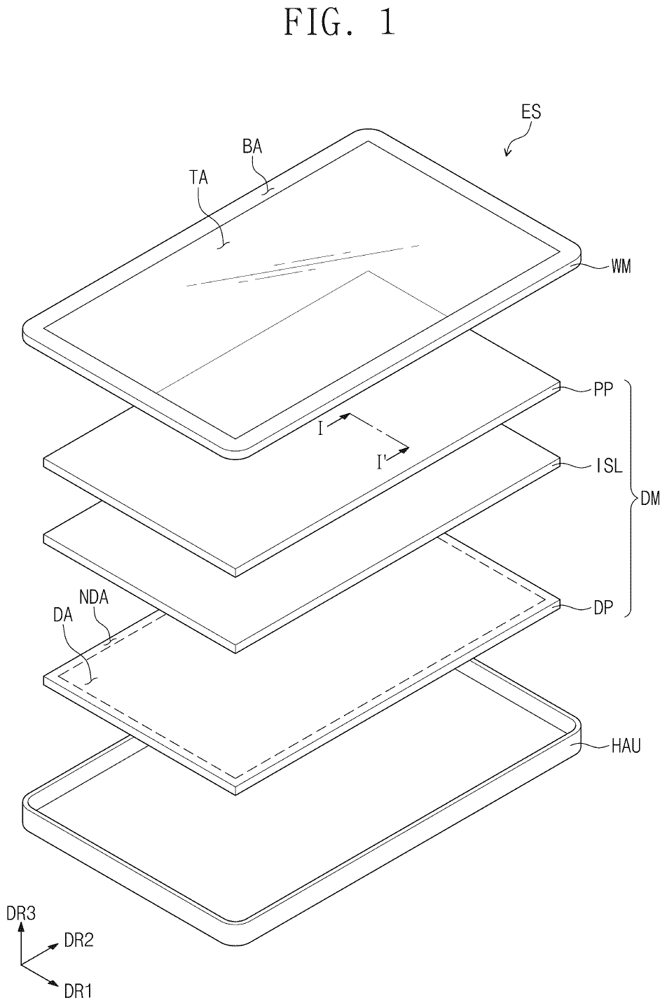

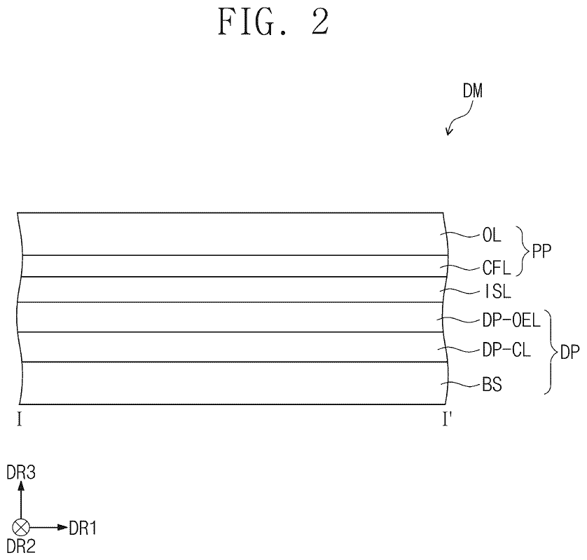

FIG. 1 is an exploded perspective view of a display device ES. FIG. 2 is a cross-sectional view of a display module DM according to an embodiment. FIG. 2 is a cross-sectional view corresponding to line I-I′ of FIG. 1.

The display device ES may be a device activated in response to electrical signals. The display device ES may include various embodiments. For example, the display device ES may not only include large-sized electronic devices such as a television set and an outdoor billboard, but also include small- and medium-sized electronic devices such as a mobile phone, a tablet computer, a navigation system, and a game console. In addition, these are merely presented as an example, and thus the display device may be adopted for other electronic devices without departing from the inventive concept.

The display device ES may display images through a front surface. The images may include still images as well as dynamic images. The front surface of the display device ES may have a flat shape or may have a curved shape.

The display device ES may be flexible. “Flexible” indicates a property of being bendable, and may include a range of bendability from completely foldable to being bendable by up to several nanometers. For example, the display device ES may be a curved display device or a foldable display device. However, the embodiment of the inventive concept is not limited thereto, and the display device ES may be rigid.

The display device ES may include a window WM, a display module DM, and a housing HAU. The display module DM may include a display panel DP, which is a display element. Meanwhile, although not shown in the drawing, the display device ES may further include various types of elements activated according to electrical signals, such as a display element, a touch element, or a detection element.

In the present description, for the convenience of description, the third direction DR3 is defined as a direction in which images are provided to users. In addition, the first direction DR1 and the second direction DR2 may be perpendicular to each other, and the third direction DR3 may be a normal direction to a plane defined by the first direction DR1 and the second direction DR2. In FIG. 1, a plane defined by the first direction DR1 and the second direction DR2 may be a display surface on which images are provided. Meanwhile, a thickness direction of the display device ES is indicated by the third direction DR3. A front surface (or an upper surface) and a rear surface (or a lower surface) of respective members are defined by the third direction DR3. However, directions indicated by the first to third directions DR1, DR2, and DR3 are relative concepts, and may thus be changed to other directions. Hereinafter, first to third directions correspond to directions indicated by the first to third directions DR1, DR2, and DR3, respectively, and are given the same reference numerals.

The display device ES may have a hexahedral shape having a thickness in the third direction DR3 on a plane defined by the first direction DR1 and the second direction DR2 crossing each other. However, this is presented as an example, and the display device ES may have various shapes and is not limited to any one embodiment.

In the display device ES according to an embodiment, the window WM may be disposed on the display module DM. The window WM may be a material including glass, sapphire, or plastic. The window WM includes a transmission region TA that transmits images provided from the display module DM, and a bezel region BA that is adjacent to the transmission region TA and does not transmit images. Meanwhile, unlike the one shown in FIG. 1, in the display device ES of an embodiment, the window WM may be omitted.

The transmission region TA may be an optically transparent region. The bezel region BA may be a portion having a relatively lower light transmittance than the transmission region TA. The bezel region BA may have a predetermined color. The bezel region BA may surround the transmission region TA. The bezel region BA may define a shape of the transmission region TA. However, the embodiment of the inventive concept is not limited to what is shown, the bezel region BA may be disposed adjacent to only one side of the transmission region TA, and a portion thereof may be omitted.

In the display device ES of an embodiment, the display module DM may be disposed below the window WM. The display module DM may include the display panel DP and the light control layer PP disposed on the display panel DP. An input sensing panel ISL may be disposed between the display panel DP and the light control layer PP. The input sensing panel ISL may be omitted.

The display panel DP may be a light emitting display panel. For example, the display panel DP may be an organic light emitting display panel, a quantum dot light emitting display panel, a micro LED display panel, or a nano LED display panel. An emission layer of the organic light emitting display panel may include an organic light emitting material. An emission layer of the quantum dot light emitting display panel may include quantum dots and/or quantum rods, and the like. The micro LED display panel may include a micro light emitting diode element, which is a subminiature light emitting element, and the nano LED display panel may include a nano light emitting diode element.

The display device ES of an embodiment may be an organic light emitting display device including an organic light emitting display panel. When viewed on a plane, one surface of the display panel DP on which images are displayed is defined as a display surface. The display surface includes a display region DA in which images are displayed and a non-display region NDA in which images are not displayed. The display region DA is defined in the center of the display panel DP when view on a plane, and may overlap the transmission region TA of the window WM.

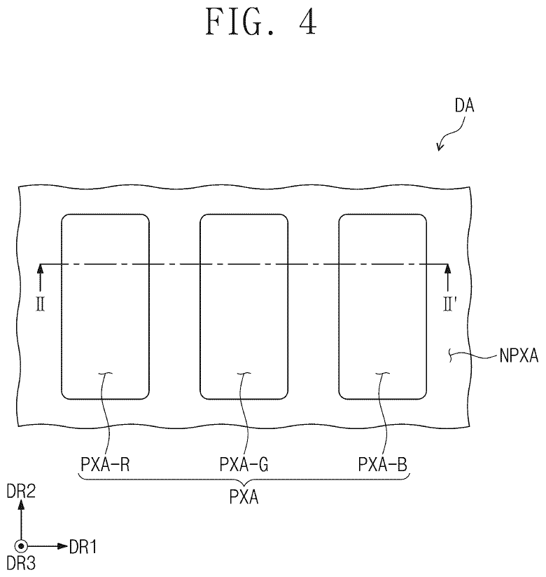

The display panel DP may include a plurality of pixels in a region corresponding to the display region DA of the display device ES. The plurality of pixels may be arranged to be spaced apart from each other in the display region DA. The plurality of pixels may display images on the display region DA by outputting light having color information in response to an electrical signal. The plurality of pixels may correspond to a light emitting region PXA (FIG. 4).

The input sensing panel ISL may be disposed on the display panel DP. The input sensing panel ISL may be bonded to the display panel DP through a separate adhesive layer. However, the embodiment of the inventive concept is not limited thereto, and the input sensing panel ISL may be directly formed on the display panel DP through a roll-to-roll process, and is not limited to any one embodiment. The input sensing panel ISL may detect external inputs through any one of a self-capacitance type or a mutual capacitance type.

The display device ES of an embodiment may further include the housing HAU. The housing HAU may be disposed under the display module DM and accommodate the display module DM. The housing HAU may be disposed to cover the display module DM such that an upper surface, which is the display surface of the display module DM is exposed. The housing HAU may cover a side surface and a bottom surface of the display module DM, and expose the whole upper surface.

Referring to FIG. 2, the display panel DP may include a base substrate BS, a circuit element layer DP-CL provided on the base substrate BS, and a display element layer DP-OEL. In an embodiment, the base substrate BS, the circuit element layer DP-CL, and the display element layer DP-OEL may be sequentially stacked in the third direction DR3.

The base substrate BS may be a member providing a base surface in which the display element layer DP-OEL is disposed. The base substrate BS may be a glass substrate, a metal substrate, a plastic substrate, etc. However, the embodiment of the inventive concept is not limited thereto, and the base substrate BS may be an inorganic layer, an organic layer, or a composite material layer. The base substrate BS may be a flexible substrate that may be readily bent or folded.

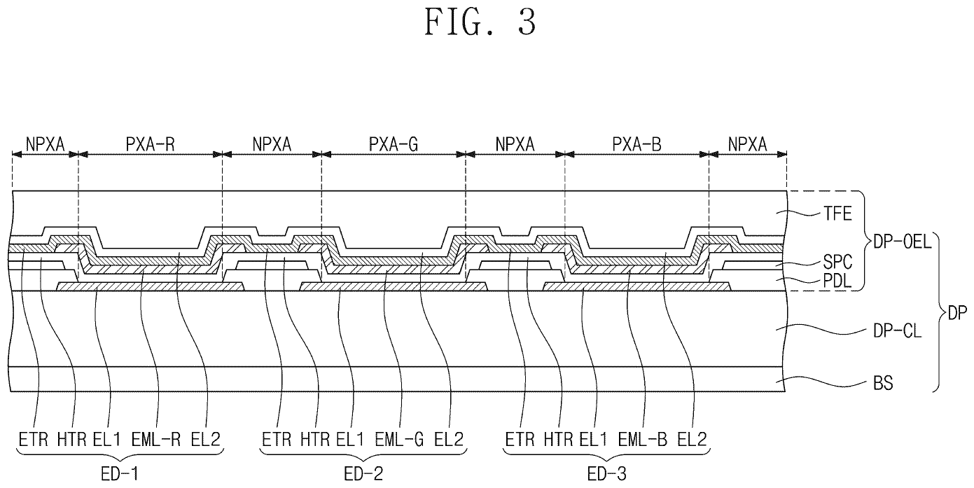

In an embodiment, the circuit element layer DP-CL may be disposed on the base substrate BS, and the circuit element layer DP-CL may include at least one transistor (not shown). The transistors (not shown) may each include a control electrode, an input electrode, and an output electrode. For example, the circuit element layer DP-CL may include a switching transistor and a driving transistor for driving a light emitting elements ED-1, ED-2, and ED-3 (FIG. 3) of the display element layer DP-OEL.

The display element layer DP-OEL may be disposed on the circuit element layer DP-CL. The display element layer DP-OEL may include a plurality of light emitting elements ED-1, ED-2, and ED-3, a pixel defining film PDL, and an encapsulation layer TFE, and a spacer SPC may also be included on at least a portion of the pixel defining film PDL. FIG. 3 is a cross-sectional view of a display panel DP according to an embodiment, which includes a spacer SPC.

The light emitting elements ED-1, ED-2, and ED-3 each include a first electrode EL1, a hole transport region HTR, emission layers EML-R, EML-G, and EML-B, an electron transport region ETR, and a second electrode EL2. The encapsulation layer TFE may be disposed on the light emitting elements ED-1, ED-2, and ED-3.

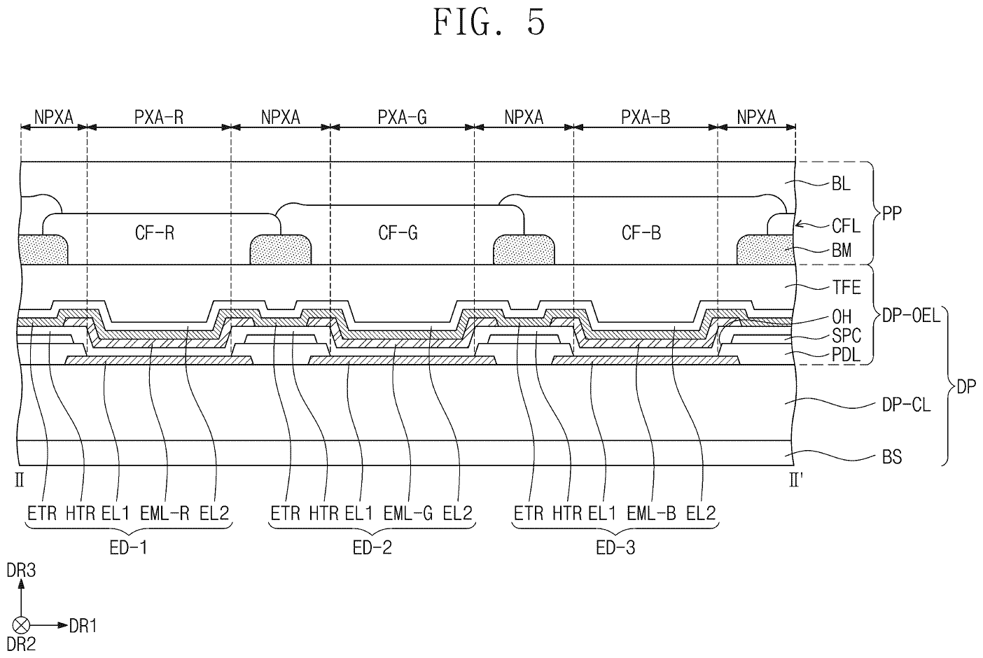

The pixel defining film PDL may be disposed on the circuit element layer DP-CL and cover a portion of the first electrode Ell. An opening OH (see FIG. 5) is defined in the pixel defining film PDL. The opening OH (FIG. 5) of the pixel defining film PDL exposes at least a portion of the first electrode EL1. In the present embodiment, light emitting regions PXA-R, PXA-G, and PXA-B are defined corresponding to a portion of the first electrode EL1 exposed through the opening OH (FIG. 5). The non-light emitting region NPXA may be disposed between the light emitting regions PXA-R, PXA-G, and PXA-B. For example, the non-light emitting region NPXA may surround the light emitting regions PXA-R, PXA-G, and PXA-B.

The spacer SPC may be disposed on a portion of the pixel defining film PDL. The pixel defining film PDL and the spacer SPC may be formed using photosensitive resin compositions having different compositions. In addition, the pixel defining film PDL and the spacer SPC may be formed using photosensitive resin compositions having the same composition.

The spacer SPC and the pixel defining film PDL may be in the form of a single body. The spacer SPC may overlap the entire pixel defining film PDL or only a portion of the pixel defining film PDL that separates the light emitting regions PXA-R, PXA-G, and PXA-B. For example, the spacer SPC may be disposed on the pixel defining film PDL with two to four of the light emitting regions PXA-R, PXA-G, and PXA-B that are adjacent in the first direction DR1 and/or the second direction DR2 therebetween. Alternatively, the spacer SPC may be disposed on the pixel defining film PDL in the non-light emitting region NPXA between adjacent two of the light emitting regions PXA-R, PXA-G, and PXA-B.

Meanwhile, although FIG. 3 shows that the spacer SPC is formed on the pixel defining film PDL, the embodiment of the inventive concept is not limited thereto. In an embodiment, the spacer SPC may be omitted from the display element layer DP-OEL.

In an embodiment, the pixel defining film PDL may be formed of a photosensitive resin composition, which will be described later.

The photosensitive resin composition according to an embodiment of the inventive concept may include a reactive unsaturated compound, an alkali soluble resin, an initiator, and a pigment, may further include a solvent, and may further include an additive when needed. The photosensitive resin composition of an embodiment may be a negative-type photosensitive resin composition in which a portion irradiated with light is insolubilized.

Hereinafter, each component included in the photosensitive resin composition according to an embodiment of the inventive concept will be described in detail.

In an embodiment, the photosensitive resin composition includes a reactive unsaturated compound. In an embodiment, the reactive unsaturated compound may include an acryl-based compound. The acryl-based compound may include at least three ethylenically unsaturated double bond groups in molecules and have a viscosity of about 200 mPa·s to about 280 mPa·s at 40° C.

When the acryl-based compound has less than 3 ethylenically unsaturated double bond groups in molecules, patterns may be hardly formed due to insufficient degree of cure upon photocuring. In addition, when the viscosity of the acryl-based compound is less than 200 mPa·s, adhesion may be degraded to reduce resolution, and when the viscosity is greater than 280 mPa·s, pattern breakup or residue may be caused.

The photosensitive resin composition according to an embodiment of the inventive concept includes, as a reactive unsaturated compound, an acryl-based compound including at least three ethylenically unsaturated double bond groups and having the viscosity described above, and may thus exhibit excellent pattern properties. To be specific, when a pixel defining film PDL is formed using the photosensitive resin composition according to an embodiment of the inventive concept, adhesion to a substrate (e.g., a circuit element layer in an embodiment) on which a pixel defining film is provided may be improved with high sensitivity upon photocuring to achieve excellent reliable patterns.

In an embodiment, the acryl-based compound may be represented by Formula 1 below.

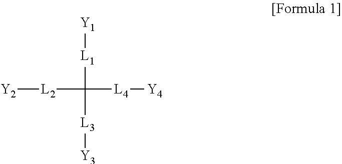

In Formula 1 above, L₁ to L₄ may each independently be a substituted or unsubstituted alkylene group having 1 to 25 carbon atoms, Y₁ to Y₄ may each independently be a hydrogen atom, a hydroxy group or a substituted or unsubstituted acrylic acid derivative, and at least three of Y₁ to Y₄ may include a substituted or unsubstituted acrylic acid derivative.

In the substituents Y₁ to Y₄ of Formula 1, the acrylic acid derivative may be acrylic acid, methacrylic acid, and a compound derived therefrom, such as acrylic acid ester and methacrylic acid ester.

As an example, the substituted or unsubstituted acrylic acid derivative of Y₁ to Y₄ may be represented by Formula 1-A below.

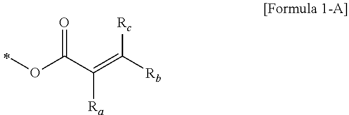

In Formula 1-A above, R a to R c may each independently be a hydrogen atom, a deuterium atom, a substituted or unsubstituted alkyl group having 1 to 20 carbon atoms, or a substituted or unsubstituted aryl group having 6 to 24 ring-forming carbon atoms. In addition, “----*” refers to a position to be connected.

In an embodiment, the acryl-based compound represented by Formula 1 above may be represented by Formula 2 or Formula 3 below.

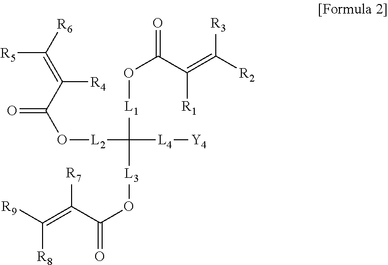

In Formula 2 above, L₁ to L₄ may be the same as defined in Formula 1 above, and may be, for example, a substituted or unsubstituted alkylene group having 1 to 25 carbon atoms. In addition, R₁ to R₉ may each independently be a hydrogen atom, a deuterium atom, a substituted or unsubstituted alkyl group having 1 to 20 carbon atoms, or a substituted or unsubstituted aryl group having 6 to 24 ring-forming carbon atoms, and Y₄ may be a hydrogen atom or a hydroxy group.

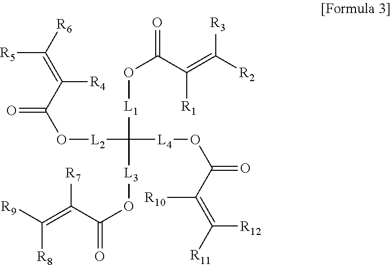

In Formula 3 above, L₁ to L₄ may be the same as defined in Formula 1 above, and may be, for example, a substituted or unsubstituted alkylene group having 1 to 25 carbon atoms. In addition, R₁ to R₁₂ may each independently be a hydrogen atom, a deuterium atom, a substituted or unsubstituted alkyl group having 1 to 20 carbon atoms, or a substituted or unsubstituted aryl group having 6 to 24 ring-forming carbon atoms.

As the acryl-based compound herein, the compound alone or combination of the two compounds represented by Formulas 1 to 3 above may be used.

The acryl-based compound may be included in an amount of about 0.3 wt % to about 30 wt %, for example, about 3 wt % to about 25 wt %, with respect to a total weight of the photosensitive resin composition. When the amount of the acryl-based compound is included in the above range, photocurability is excellent and thus pattern properties are excellent.

In an embodiment, the reactive unsaturated compound may further include a monomer and/or an oligomer generally usable in the photosensitive resin composition as a material that may be used together with the acryl-based compound. For example, the reactive unsaturated compound may further include at least one of a monomer or an oligomer, which has at least one ethylenically unsaturated double bond. For example, the monomer and/or oligomer may be a monofunctional or multifunctional ester of (meth)acrylic acid having at least one ethylenically unsaturated double bond.

The photosensitive resin composition according to an embodiment of the inventive concept may further include a monomer and/or an oligomer having an ethylenically unsaturated double bond together with the acryl-based compound to cause sufficient polymerization upon exposure to light in a pattern forming process, thereby forming patterns having excellent heat resistance, light resistance, and chemical resistance.

For example, the monomer or the oligomer may specifically be ethylene glycol diacrylate, ethylene glycol dimethacrylate, diethylene glycol diacrylate, triethylene glycol diacrylate, triethylene glycol dimethacrylate, 1,6-hexanediol diacrylate, 1,6-hexanediol dimethacrylate, pentaerythritol triacrylate, dipentaerythritol pentaacrylate, dipentaerythritol hexaacrylate, bisphenol A epoxy acrylate, ethylene glycol monomethyl ether acrylate, trimethylolpropane triacrylate, trisacryloyloxyethyl phosphate, and the like.

Examples of commercially available products of the monomer or the oligomer are as follows.

Examples of the monofunctional ester of (meth)acrylic acid may include Aronix M-101, M-111, and M-114 (manufactured by Toagosei Kagaku Kogyo Co., Ltd.), KAYARAD TC-110S® and TC-120S (manufactured by Nippon Kayaku Co., Ltd.), V-158 and V-2311 (manufactured by Osaka Yuki Kagaku Kogyo Co., Ltd.), and the like. Examples of the bifunctional ester of (meth)acrylic acid may include Aronix M-210, M-240, and M-6200 (manufactured by Toagosei Kagaku Kogyo Co., Ltd.), KAYARAD HDDA, HX-220, and R-604 (manufactured by Nippon Kayaku Co., Ltd.), V-260, V-312, and V-335 HP (manufactured by Osaka Yuki Kagaku Kogyo Co., Ltd.), and the like. Examples of the trifunctional ester of (meth)acrylic acid may include Aronix M-309, M-400, M-405, M-450, M-7100, M-8030, and M-8060 (manufactured by Toagosei Kagaku Kogyo Co., Ltd.), KAYARAD TMPTA, DPCA-20, DPCA-60, and DPCA-120 (manufactured by Nippon Kayaku Co., Ltd.), V-295, V-300, and V-360 (manufactured by Osaka Yuki Kayaku Kogyo Co., Ltd.), and the like. These products may be used alone or in combination of two or more.

The reactive unsaturated compound may be used after being treated with an acid anhydride to improve developability.

When the photosensitive resin composition according to an embodiment of the inventive concept includes the monomer and/or the oligomer, the monomer and/or the oligomer may be included in an amount of about 1 wt % to 40 wt %, specifically about 1 wt % to about 20 wt %, with respect to a total weight of the photosensitive resin composition. When the photosensitive resin composition includes a monomer and/or an oligomer within the above range, in the pattern forming process, curing sufficiently takes place upon exposure, resulting in excellent reliability, and excellent heat resistance, light resistance, and chemical resistance of patterns, and excellent resolution and adhesiveness as well.

In an embodiment, the photosensitive resin composition according to an embodiment of the inventive concept includes an alkali-soluble resin.

In an embodiment, the alkali soluble resin may include a cardo-based resin, an acryl-based resin, a polyester-based resin, a polyurethane-based resin, a polysiloxane resin, a polycyclic side chain-containing resin, or an acid-modified epoxy resin alone or may include two or more resins selected from these resins.

The cardo-based resin may include a repeating unit represented by Formula 4 below.

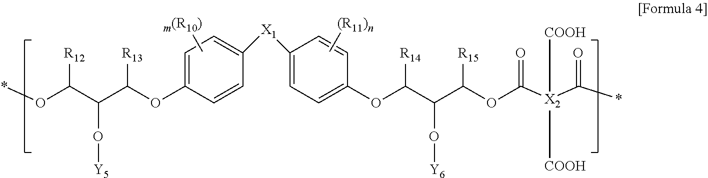

In Formula 4, “*” is a mark indicating a portion where bonds are connected by a repeating unit.

In addition, in Formula 4, m and n may each independently be an integer of 0 to 4. R₁₀ and R₁₁ may each independently be a deuterium atom, a halogen atom, or a substituted or unsubstituted C₁-C₂₀ alkyl group (when m and n are 0, hydrogen bonded to benzene is omitted).

In Formula 4, R₁₂ to R₁₅ may each independently be a hydrogen atom, a deuterium atom, a halogen atom, or a substituted or unsubstituted C₁-C₂₀ alkyl group.

For example, X₁ in Formula 4 is may be any one among a direct linkage, O, CO, SO₂, CR′R″, SiR′R″ (where R′ and R″ are each independently a hydrogen atom or a substituted or unsubstituted C₁-C₂₀ alkyl group), or a compound represented by Formula 5.

In Formula 4, X₂ may be an acid anhydride residue or an acid dianhydride residue, and Y₅ and Y₆ may each independently be a hydrogen atom, a deuterium atom, or represented by Formula 6 below. In addition, at least one of Y₅ or Y₆ is represented by Formula 6 below.

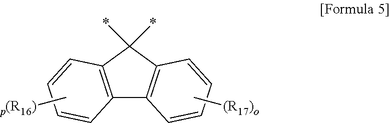

In Formula 5, R₁₆ and R₁₇ may each independently be a deuterium atom, a halogen atom, a substituted or unsubstituted C₁-C₂₀ alkyl group, a substituted or unsubstituted C₁-C₂₀ alkoxy group, or a substituted or unsubstituted C₁-C₂₀ alkenyl group (when o and p are 0, hydrogen bonded to benzene is omitted), and o and p may each independently be an integer from 0 to 4.

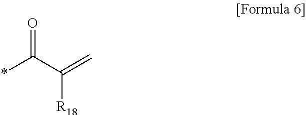

In Formula 6, R₁₈ may be a hydrogen atom, a deuterium atom, or a methyl group.

In an embodiment, the cardo-based resin may have a weight average molecular weight of about 1,000 g/mol to about 100,000 g/mol. For example, in an embodiment, the cardo-based resin may have a weight average molecular weight of about 1,000 g/mol to about 50,000 g/mol, and specifically, the cardo-based resin may have a weight average molecular weight of about 1,000 g/mol to about 20,000 g/mol. When the weight average molecular weight of the cardo-based resin is within the above range, patterns may be well formed without residue upon manufacture of a pixel defining film. In addition, when the weight average molecular weight of the cardo-based resin is within the above range, there is no loss of film thickness upon developing, and satisfactory patterns may be obtained.

In the case of using the cardo-based resin as an alkali-soluble resin herein, the cardo-based resin may be included in an amount of about 1 wt % to about 30 wt %, for example, about 3 wt % to about 20 wt %, with respect to a total weight of the photosensitive resin composition. When the cardo-based resin is included within the above range, excellent sensitivity, developability, and adhesion (or adhesiveness) may be obtained.

The acryl-based resin may be a copolymer including at least one repeating unit of Formulas 7 to 9 below.

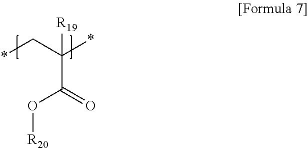

In Formula 7, R₁₉ may be a hydrogen atom, a deuterium atom, or a methyl group, R₂₀ may be a hydrogen atom, a substituted or unsubstituted C₁-C₂₀ alkyl group, a substituted or unsubstituted aryl group having 6 to 20 ring-forming carbon atoms, a substituted or unsubstituted ring-forming C₃-C₂₀ cycloalkyl group, a substituted or unsubstituted bis- or tri-cycloalkyl group having 7 to 20 ring-forming carbon atoms, or a substituted or unsubstituted bis- or tri-cycloaryl group having 7 to 20 ring-forming carbon atoms. In an embodiment, when the acryl-based resin includes two or more repeating units of Formula 7, at least one R₂₀ may be hydrogen.

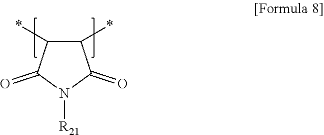

In Formula 8, R₂₁ may be a substituted or unsubstituted C₁-C₂₀ alkyl group, a substituted or unsubstituted ring-forming C₆-C₂₀ aryl group, or a substituted or unsubstituted ring-forming C₆-C₂₀ cycloalkyl group.

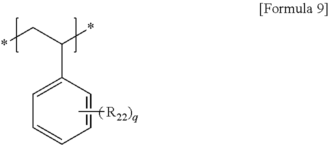

In Formula 9, R₂₂ may be a methyl group, a sulfonic acid group, a nitro group, a substituted or unsubstituted amino group, or a hydroxy group, and q may be an integer from 0 to 5.

The acryl-based resin may have a weight average molecular weight of about 3,000 g/mol to about 50,000 g/mol, for example, about 5,000 g/mol to about 30,000 g/mol, specifically about 7,000 g/mol to about 20,000 g/mol. When the weight average molecular weight of the acryl-based resin is within the above range, patterns may be well formed without breakup or residue upon manufacture of a pixel defining film PDL.

In an embodiment, the acryl-based resin may be included in the range of about 0 wt % to about 10 wt % with respect to a total weight of the photosensitive resin composition. In the present description, development rate and taper angle may be regulated by adjusting the amount of the acryl-based resin to the above range.

Types of the polyester-based resin, polyurethane-based resin, polysiloxane resin, polycyclic side chain-containing resin, and/or acid-modified epoxy resin are not particularly limited, and resins usable in the photosensitive resin composition may be used.

The photosensitive resin composition according to an embodiment of the inventive concept includes an initiator.

The initiator may include a photopolymerization initiator or a radical polymerization initiator, or a combination thereof.

The photopolymerization initiator is an initiator generally used in the photosensitive resin composition, and for example, the photopolymerization initiator may include an acetophenone-based compound, a benzophenone-based compound, a thioxanthone-based compound, a benzoin-based compound, an oxime ester-based compound, a triazine-based compound, and the like.

The acetophenone-based compound may include, for example, 2,2′-diethoxy acetophenone, 2,2′-dibutoxy acetophenone, 2-hydroxy-2-methylpropiophenone, p-t-butyltrichloro acetophenone, p-t-butyldichloro acetophenone, 4-chloro acetophenone, 2,2′-dichloro-4-phenoxy acetophenone, 2-methyl-1-(4-(methylthio)phenyl)-2-morpholinopropane-1-one, 2-benzyl-2-dimethylamino-1-(4-morpholinophenyl)-butan-1-one, and the like.

The benzophenone-based compound may include, for example, benzophenone, benzoyl benzoate, methyl benzoyl benzoate, 4-phenyl benzophenone, hydroxy benzophenone, acrylated benzophenone, 4,4′-bis(dimethylamino)benzophenone, 4,4′-bis(diethylamino)benzophenone, 4,4′-dimethylaminobenzophenone, 4,4′-dichlorobenzophenone, 3,3′-dimethyl-2-methoxybenzophenone, and the like.

The thioxanthone-based compound may include, for example, thioxanthone, 2-chlorothioxanthone, 2-methylthioxanthone, isopropyl thioxanthone, 2,4-diethyl thioxanthone, 2,4-diisopropyl thioxanthone, 2-chlorothioxanthone, and the like.

The benzoin-based compounds may include, for example, benzoin, benzoin methylether, benzoin ethylether, benzoin isopropyl ether, benzoin isobutyl ether, and benzyl dimethylketal.

The triazine-based compound may include, for example, 2,4,6-trichloro-s-triazine, 2-phenyl 4,6-bis(trichloromethyl)-s-triazine, 2-(3′, 4′-dimethoxystyryl)-4,6-bis(trichloromethyl)-s-triazine, 2-(4′-methoxynaphthyl)-4,6-bis(trichloromethyl)-s-triazine, 2-(p-methoxyphenyl)-4,6-bis(trichloromethyl)-s-triazine, 2-(p-tolyl)-4,6-bis(trichloromethyl)-s-triazine, 2-biphenyl 4,6-bis(trichloromethyl)-s-triazine, bis(trichloromethyl)-6-styryl-s-triazine, 2-(naphtho 1-yl)-4,6-bis(trichloromethyl)-s-triazine, 2-(4-methoxynaphtho 1-yl)-4,6-s(trichloromethyl)-s-triazine, 2-4-trichloromethyl(piperonyl)-6-triazine, 2-4-trichloromethyl(4′-methoxystyryl)-6-triazine, and the like.

The oxime ester-based compound may include, for example, o-ethoxycarbonyl-α-oxyimino-1-phenylpropane-1-one, and may include OXE-01, OXE-02, and the like from BASF as commercially available products.

The photopolymerization initiator may include a carbazole-based compound, a diketone-based compound, a sulfonium borate-based compound, a diazo-based compound, an imidazole-based compound, a biimidazole-based compound, and the like, in addition to the above compounds.

The radical polymerization initiator may include a peroxide-based compound or an azobis-based compound.

The peroxide-based compound may include, for example, ketone peroxides such as methyl ethyl ketone peroxide, methyl isobutyl ketone peroxide, cyclohexanone peroxide, methylcyclohexanone peroxide, and acetylacetone peroxide; diacyl peroxides such as isobutyryl peroxide, 2,4-dichlorobenzoyl peroxide, o-methylbenzoyl peroxide, and bis-3,5,5-trimethylhexanoyl peroxide; hydroperoxides such as 2,4,4-trimethylpentyl-2-hydroperoxide, diisopropylbenzene hydroperoxide, cumene hydroperoxide, and t-butyl hydroperoxide; dialkyl peroxides such as dicumyl peroxide, 2,5-dimethyl-2,5-di(t-butylperoxy)hexane, 1,3-bis(t-butyloxyisopropyl)benzene, and t-butylperoxyvaleric acid n-butyl ester; alkyl peresters such as 2,4,4-trimethylpentyl peroxyphenoxyacetate, α-cumyl peroxyneodecanoate, t-butyl peroxybenzoate, and di-t-butyl peroxytrimethyl adipate; percarbonates such as di-3-methoxy butyl peroxydicarbonate, di-2-ethylhexyl peroxydicarbonate, bis-4-t-butylcyclohexyl peroxydicarbonate, diisopropyl peroxydicarbonate, acetylcyclohexylsulfonyl peroxide, and t-butyl peroxyaryl carbonate, and the like.

The azobis-based compound may include, for example, 1,1′-azobiscyclohexane-1-carbonitrile, 2,2′-azobis(2,4-dimethylvaleronitrile), 2,2,-azobis(methylisobutyrate), 2,2′-azobis(4-methoxy-2,4-dimethylvaleronitrile), α, α′-azobis(isobutylnitrile), 4,4′-azobis(4-cyanovaleric acid), and the like.

The radical polymerization initiator may be used together with a photosensitizer that causes chemical reactions by absorbing light to become excited and then transferring energy.

The photosensitizer may include, for example, tetraethylene glycol bis-3-mercapto propionate, pentaerythritol tetrakis-3-mercapto propionate, dipentaerythritol tetrakis-3-mercapto propionate, and the like.

In an embodiment, the initiator may be included in an amount of about 0.01 wt % to about 10 wt %, specifically about 0.1 wt % to about 5 wt %, with respect to a total weight of the photosensitive resin composition. When the initiator is included in the photosensitive resin composition within the above range, in the pattern forming process, curing sufficiently takes place upon exposure, resulting in excellent reliability. In addition, excellent resolution and adhesiveness as well as excellent heat resistance, light resistance, and chemical resistance of patterns may be provided, and a decrease in transmittance due to unreacted initiators may be prevented.

The photosensitive resin composition of an embodiment includes a pigment. In an embodiment, the pigment may be an organic pigment and/or an inorganic pigment.

As the pigment, a red pigment, a green pigment, a blue pigment, a yellow pigment, a black pigment, and the like may be used. In an embodiment, the pigment may be a compound specifically classified as a pigment in a color index (published by The Society of Dyers and Colourists). For example, the pigment may include pigments referred to by color index (C.I.) numbers below, but the embodiment of the inventive concept is not limited thereto.

For example, the red pigment may include C.I. red pigment 254, C.I. red pigment 255, C.I. red pigment 264, C.I. red pigment 270, C.I. red pigment 272, C.I. red pigment 177, C.I. red pigment 89, and the like.

The green pigment may include halogen-substituted copper phthalocyanine pigments such as C.I. green pigment 36 and C.I. green pigment 7.

The blue pigment may include copper phthalocyanine pigments such as C.I. blue pigment 15:6, C.I. blue pigment 15, C.I. blue pigment 15:1, C.I. blue pigment 15:2, C.I. blue pigment 15:3, C.I. blue pigment 15:4, C.I. blue pigment 15:5, and C.I. blue pigment 16.

The yellow pigment may include isoindoline-based pigments such as C.I. yellow pigment 139, quinophthalone-based pigments such as C.I. yellow pigment 138, nickel complex pigments such as yellow pigment 150.

The black pigment may include organic black pigments such as benzofuranone-based black pigments, azo-based black pigments, indolinone-based black pigments, lactam black, aniline black, and perylene-based black pigments, and inorganic black pigments such as titanium black and carbon black.

These pigments may be used alone or in combination of two or more. The pigment of an embodiment may include a black organic pigment, a black inorganic pigment, and a mixture of two or more coloring pigments. For example, the photosensitive resin composition of an embodiment may use a black pigment alone as a pigment or a mixture of two or more kinds of coloring pigments such as the red, green, blue, yellow, and/or black pigments, but the embodiment of the inventive concept is not limited thereto.

In an embodiment, a pixel defining film PDL may be a black pixel defining film. In an embodiment, the pixel defining film PDL may be formed by including a black pigment or the like. For example, the pixel defining film PDL may be formed by including at least one of a benzofuranone-based black pigment, a perylene-based black pigment, an azo-based black pigment, and an indolinone-based black pigment. The black pixel defining film may absorb light reflected by a metal layer or the like in a circuit element or light reflected by external light to improve display quality of a display device ES.

In an embodiment, the black organic pigment may further include a coating layer on a surface. The coating layer may include one or more types selected from the group consisting of a silica coating layer, a metal oxide coating layer, and a metal hydroxide coating layer. The black organic pigment containing the coating layer has excellent dispersion stability and provides electrical benefits such as low permittivity.

When the black pigment is used as a pigment included in the photosensitive resin composition according to an embodiment of the inventive concept, the black pigment may be used together with color correcting agents such as an anthraquinone-based pigment, a perylene-based pigment, a phthalocyanine-based pigment, or an azo-based pigment.

In an embodiment, a dispersant may be used together to disperse the pigment in the photosensitive resin composition. To be specific, the pigment may be subjected to surface treatment in advance with a dispersant and used, or a dispersant may be added together with the pigment upon preparation of the photosensitive resin composition.

Nonionic dispersants, anionic dispersants, cationic dispersants, and the like may be used as the dispersant, but the dispersant is not limited thereto. Specifically, the dispersant may include, for example, polyalkylene glycol and ester thereof, polyoxyalkylene, polyhydric alcohol ester alkylene oxide adducts, alcoholalkylene oxide adducts, sulfonic acid ester, sulfonic acid salt, carboxylic acid ester, carboxylic acid salt, alkylamide alkylene oxide adducts, alkylamine, and the like, and these may be used alone or in combination of two or more.

Commercially available products of the dispersant may include, for example, DISPERBYK-101, DISPERBYK-130, DISPERBYK-140, DISPERBYK-160, DISPERB YK-161, DISPERBYK-162, DISPERB YK-163, DISPERB YK-164, DISPERB YK-165, DISPERBYK-166, DISPERBYK-170, DISPERB YK-171, DISPERBYK-182, DISPERBYK-2000, DISPERBYK-2001, and the like (manufactured by BYK), EFKA-47, EFKA-47EA, EFKA-48, EFKA-49, EFKA-100, EFKA-400, and EFKA-450 (manufactured by BASF), Solsperse 5000, Solsperse 12000, Solsperse 13240, Solsperse 13940, Solsperse 17000, Solsperse 20000, Solsperse 24000GR, Solsperse 27000, Solsperse 28000, and the like (manufactured by Zeneka), or PB711, PB821, and the like (manufactured by Ajinomoto).

The dispersant may be included in an amount of about 0.1 wt % to about 15 wt % with respect to a total weight of the photosensitive resin composition. When the dispersant is included within the above range, the photosensitive resin composition has excellent dispersibility and thus stability, developability, and pattern properties are excellent when manufacturing the pixel defining film PDL.

The pigment may be used after being pretreated with a water-soluble inorganic salt and a wetting agent. When the pigment is used after being subjected to the pretreatment, primary particle size of the pigment may be refined. The pretreatment may be performed by kneading the pigment with a water-soluble inorganic salt and a wetting agent, and filtering and washing the pigment obtained from the kneading. The kneading may be performed at a temperature of about 40° C. to about 100° C., and the filtration and washing may be performed by washing the inorganic salt with water and then filtering.

The water-soluble inorganic salt may include, for example, sodium chloride, potassium chloride, and the like, but is not limited thereto.

The wetting agent serves as a medium in which the pigment and the water-soluble inorganic salt are uniformly mixed so that the pigment may be easily pulverized, and may include, for example, alkylene glycol monoalkyl ether such as ethylene glycol monoethyl ether, propylene glycol monomethyl ether, and diethylene glycol monomethyl ether; and alcohol such as ethanol, isopropanol, butanol, hexanol, cyclohexanol, ethylene glycol, diethylene glycol, polyethylene glycol, glycerin, and polyethylene glycol, and these may be used alone or in combination of two or more.

The pigment subjected to the kneading may have an average particle diameter of about 20 nm to about 110 nm. When the average particle diameter of the pigment is within the above range, fine patterns having excellent heat resistance and light resistance may be effectively formed.

In an embodiment, the pigment may be included in an amount of about 1 wt % to about 30 wt % with respect to a total weight of the photosensitive resin composition. When the pigment is included within the above range, reflection caused by light entering an element from the outside is well blocked, and accordingly, a display device ES to which a pixel defining film PDL according to an embodiment of the inventive concept is applied has excellent color reproducibility and excellent pattern curability and adhesion.

The photosensitive resin composition of an embodiment may include a solvent.

As the solvent, materials that have compatibility with but do not react with the alkali-soluble resin, the reactive unsaturated compound, the pigment, and the initiator may be used.

In the photosensitive resin composition according to an embodiment of the inventive concept, the solvent may be alcohol such as methanol and ethanol; ether such as dichloroethyl ether, n-butyl ether, diisoamyl ether, methylphenyl ether, tetrahydrofuran, ethylene glycol monomethyl ether and ethylene glycol monoethyl ether; cellosolve acetate such as methyl cellosolve acetate, ethyl cellosolve acetate, and diethyl cellosolve acetate; carbitol such as methyl ethyl carbitol, diethyl carbitol, diethylene glycol monomethyl ether, diethylene glycol monoethyl ether, diethylene glycol dimethyl ether, diethylene glycol methyl ethyl ether, and diethylene glycol diethyl ether; propylene glycol alkyl ether acetate such as propylene glycol methyl ether acetate and propylene glycol propyl ether acetate; aromatic hydrocarbon such as toluene and xylene; ketone such as methyl ethyl ketone, cyclohexanone, 4-hydroxy-4-methyl-2-pentanone, methyl-n-propyl ketone, methyl-n-butyl ketone, methyl-n-amyl ketone, and 2-heptanone; saturated aliphatic monocarboxylic acid alkyl ester such as ethyl acetate, n-butyl acetate, and isobutyl acetate; lactic acid ester such as methyl lactate and ethyl lactate; oxyacetic acid alkyl ester such as methyl oxyacetate, ethyl oxyacetate, and butyl oxyacetate; alkoxy acetic acid alkyl ester such as methoxy methyl acetate, methoxy ethyl acetate, methoxy butyl acetate, ethoxy methyl acetate, and ethoxy ethyl acetate; 3-oxy propionic acid alkyl ester such as 3-oxy methyl propionate and 3-oxy ethyl propionate; 3-alkoxy propionic acid alkyl ester such as 3-methoxy methyl propionate, 3-methoxy ethyl propionate, 3-ethoxy ethyl propionate, and 3-ethoxy methyl propionate; 2-oxypropionic acid alkyl ester such as methyl 2-oxypropionate, ethyl 2-oxypropionate, and propyl 2-oxypropionate; 2-alkoxy propionic acid alkyl ester such as 2-methoxy methyl propionate, 2-methoxy ethyl propionate, 2-ethoxy ethyl propionate, and 2-ethoxy methyl propionate; 2-oxy-2-methyl propionic acid ester such as 2-oxy-2-methyl methyl propionate and 2-oxy-2-methyl ethyl propionate; monooxy monocarboxylic acid alkyl ester of 2-alkoxy-2-methyl alkyl propionate such as 2-methoxy-2-methyl methyl propionate and 2-ethoxy-2-methyl ethyl propionate; ester such as 2-hydroxy ethyl propionate, 2-hydroxy-2-methyl ethyl propionate, hydroxy ethyl acetate, and 2-hydroxy-3-methyl methyl butanoate; keto acid ester such as ethyl pyruvate, and the like, and in addition, may include a solvent having a high boiling point such as N-methylformamide, N,N-dimethylformamide, N-methylformanilide, N-methylacetamide, N,N-dimethylacetamide, N-methylpyrrolidone, dimethylsulfoxide, benzylethyl ether, dihexyl ether, acetyl acetone, isophorone, caproic acid, caprylic acid, 1-octanol, 1-nonanol, benzyl alcohol, benzyl acetate, ethyl benzoate, diethyl oxalate, diethyl maleate, γ-butyrolactone, ethylene carbonate, propylene carbonate, phenyl cellosolve acetate, and the like.

For example, among the solvents, considering compatibility and reactivity, glycol ether such as ethylene glycol monoethyl ether; ethylene glycol alkyl ether acetate such as ethyl cellosolve acetate; ester such as 2-hydroxy ethyl propionate; carbitol such as diethylene glycol monomethyl ether; propylene glycol alkyl ether acetate such as propylene glycol methyl ether acetate, and propylene glycol propyl ether acetate may be included.

In an embodiment, the solvent may be included in a balance amount with respect to a total weight of the photosensitive resin composition.

The balance amount indicates a remaining amount such that the weight of a total composition further including essential components and other components of a photosensitive resin composition is 100 wt %. For example, the solvent may be included in an amount of about 50 wt % to about 90 wt % with respect to a total weight of the photosensitive resin composition. When the solvent is included in the photosensitive resin composition according to an embodiment of the inventive concept within the above range, the photosensitive resin composition has an appropriate viscosity, and processability may thus be excellent when manufacturing a pixel defining film PDL.

In an embodiment, the pixel defining film PDL may be formed through a photolithography process using a photomask after applying a photosensitive resin composition onto a circuit element layer DP-CL and a first electrodes ELL In this case, the photosensitive resin composition described above may be applied to form the pixel defining film PDL.

The pixel defining film PDL is formed on the circuit element layer DP-CL, and an opening OH is defined in the pixel defining film PDL to expose an upper surface of the first electrode EL1, using a photolithography process.

In an embodiment, the photolithography process may include a coating layer forming step of applying the photosensitive resin composition onto the circuit element layer DP-CL and the first electrode EL1 to form a coating layer. In this case, the applying may be performed through a wet coating method using an applying device such as a roll coater, a spin coater, a slit and spin coater, a slit coater (also referred to as a die coater), or inkjet to obtain a desired thickness.

When needed, a prebaking step of heating the formed coating film using an oven, a hot plate, or the like may be further included. Heating temperature and heating time upon the prebaking are selected according to a solvent used, and the prebaking may be performed, for example, for a period of about 1 minute to about 30 minutes at a temperature of about 80° C. to less than 100° C.

In addition, the photolithography process may include an exposure step of performing irradiation of activated actinic rays such as visible light, ultraviolet rays, X-rays, and electron rays, using a photomask. The photomask may include a transmission region, a blocking region, and a transflective region of the activated actinic rays. Accordingly, the exposed portion of the coating film remains in the developing step, and the unexposed portion is removed through the developing step.

The photolithography process may include a developing step of forming patterns of the coating film through a process of developing using a developer after the exposure step. An alkaline developer may be used as the developer. The developer may be used in combination with a surfactant, an antifoaming agent, an organic base, water, or the like.

The photolithography process of an embodiment includes a post-processing step. The post-treatment step is a process of heating the patterns formed through the developing step to obtain cured patterns of a photosensitive resin composition, and for example, post-baking may be performed in the step.

The post-baking may increase adhesiveness between a patterned film and a lower substrate, improve heat resistance, light resistance, crack resistance, and chemical resistance of the patterned film, and provide the patterned film with characteristics such as high strength. The post-baking may be performed using an oven, a hot plate, or the like, in the same manner as the pre-baking.

FIG. 4 is a plan view enlarging and showing a portion of a display region DA shown in FIG. 1. FIG. 5 is a cross-sectional view of a display module DM (FIG. 2) according to an embodiment, corresponding to line II-IT of FIG. 4. In FIG. 5, the input sensing panel ISL (FIG. 2) is omitted.

In the description of FIGS. 4 and 5, duplicated descriptions as one described above in FIGS. 1 to 3 will not be given again, and differences will be mainly described.

The display region DA may include a light emitting region PXA and a non-light emitting region NPXA adjacent to the light emitting region PXA.

FIG. 4 mainly shows three light emitting regions PXA-R, PXA-G, and PXA-B. The three light emitting regions PXA-R, PXA-G, and PXA-B shown in FIG. 4 may be repeatedly disposed throughout the display region DA. The non-light emitting region NPXA may set a border between the first to third light emitting regions PXA-R, PXA-G, and PXA-B to prevent the first to third light emitting regions PXA-R, PXA-G, and PXA-B) from being color mixed.

In an embodiment, the first to third light emitting regions PXA-R, PXA-G, and PXA-B having the same planar area are shown, but the embodiment of the inventive concept is not limited thereto. The first to third light emitting regions PXA-R, PXA-G, and PXA-B may have different areas or only some of the first to third light emitting regions PXA-R, PXA-G, and PXA-B may have different areas. The shapes of the first to third light emitting regions PXA-R, PXA-G, and PXA-B are not limited to the illustrated rectangular shape and may have a polygonal shape. In this case, the area may refer to an area when viewed on a plane defined by the first direction DR1 and the second direction DR2.

Each of the first to third light emitting regions PXA-R, PXA-G, and PXA-B may correspond to a pixel. Each of the first to third light emitting regions PXA-R, PXA-G, and PXA-B may provide different color light in response to electrical signals. Accordingly, a plurality of pixels corresponding to each of the first to third light emitting regions PXA-R, PXA-G, and PXA-B may output light having color information.

One of the first to third light emitting regions PXA-R, PXA-G, and PXA-B may provide first light to users, another may provide second light different from the first light, and the other may provide third light different from the first light and the second light. In an embodiment, the first light emitting region PXA-R may emit red light, the second light emitting region PXA-G may emit green light, and the third light emitting region PXA-B may emit blue light. The green light may be light having a wavelength of about 495 nm to about 570 nm, but is not limited thereto and may include wavelength ranges that may be recognized as green. The red light may be light having a wavelength of about 620 nm to about 750 nm, but is not limited thereto and may include wavelength ranges that may be recognized as red.

The display module DM of an embodiment may include a display panel DP having a plurality of light emitting elements ED-1, ED-2, and ED-3 and a light control layer PP disposed on the display panel DP. In addition, unlike what is shown, the input sensing panel ISL (FIG. 2) may be included between the display panel DP and the light control layer PP.

Referring to FIGS. 4 and 5, the light emitting regions PXA-R, PXA-G, and PXA-B each may be a region emitting light generated from each of the light emitting elements ED-1, ED-2, and ED-3. The light emitting regions PXA-R, PXA-G, and PXA-B may be spaced apart from each other when viewed on a plane.

The plurality of light emitting elements ED-1, ED-2, and ED-3 may emit light having different wavelength ranges. For example, in an embodiment, the display module DM may include a first light emitting element ED-1 emitting red light, a second light emitting element ED-2 emitting green light, and a third light emitting element ED-3 emitting blue light. However, the embodiment of the inventive concept is not limited thereto, and the first to third light emitting elements ED-1, ED-2 and ED-3 may emit light in the same wavelength range or emit light in at least one different wavelength range.

For example, the first light emitting region PXA-R, the second light emitting region PXA-G, and the third light emitting region PXA-B of the display module DM may correspond to the first light emitting element ED-1, the second light emitting element ED-2, and the third light emitting element ED-3, respectively.

The light emitting elements ED-1, ED-2, and ED-3 may have the same structure. However, in the light emitting elements ED-1, ED-2, and ED-3 of an embodiment, emission layers EML-R, EML-G, and EML-B may include different light emitting materials.

Hereinafter, the first light emitting element ED-1 will be described as an example of the light emitting element of an embodiment. The light emitting element ED-1 according to an embodiment includes a first electrode EL1, a second electrode EL2 facing the first electrode EL1, and a plurality of functional layers disposed between the first electrode EL1 and the second electrode EL2 and having an emission layer EML-R.

The plurality of functional layers may include a hole transport region HTR disposed between the first electrode EL1 and the emission layer EML-R, and an electron transport region ETR disposed between the second electrode EL2 and the emission layer EML-R. Meanwhile, although not shown in the drawings, in an embodiment, a capping layer may be further disposed on the second electrode EL2.

The hole transport region HTR and the electron transport region ETR each may include a plurality of sub functional layers. For example, the hole transport region HTR may include a hole injection layer and a hole transport layer as a sub functional layer, and the electron transport region ETR may include an electron injection layer and an electron transport layer as a sub functional layer. Meanwhile, the embodiment of the inventive concept is not limited thereto, and the hole transport region HTR may further include an electron blocking layer as a sub functional layer, and the electron transport region ETR may further include a hole blocking layer as a sub functional layer.

In the light emitting element ED-1 according to an embodiment, the first electrode EL1 has conductivity. The first electrode EL1 may be formed of a metal alloy or a conductive compound. The first electrode EL1 may be an anode. The first electrode EL1 may be a pixel electrode.

In the light emitting element ED-1 according to an embodiment, the first electrode EL1 may be an anode or a cathode. However, the embodiment of the inventive concept is not limited thereto. In addition, the first electrode EL1 may be a pixel electrode. The first electrode EL1 may be a transmissive electrode, a transflective electrode, or a reflective electrode. When the first electrode EL1 is the transmissive electrode, the first electrode EL1 may include a transparent metal oxide such as indium tin oxide (ITO), indium zinc oxide (IZO), zinc oxide (ZnO), and indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, W, a compound thereof, or a mixture thereof (e.g., a mixture of Ag and Mg). Alternatively, the first electrode EL1 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), and the like. For example, the first electrode EL1 may be a multilayer metal film and may have a structure in which metal films of ITO/Ag/ITO are stacked. The first electrode EL1 may have a thickness of about 700 Å to about 10000 Å. For example, the first electrode EL1 may have a thickness of 1000 Å to about 3000 Å.

The hole transport region HTR is provided on the first electrode EL1. The hole transport region HTR may include a hole injection layer, a hole transport layer, etc. In addition, the hole transport region HTR may further include at least one of a hole buffer layer or an electron blocking layer in addition to the hole injection layer and the hole transport layer. The hole buffer layer may compensate a resonance distance according to the wavelength of light emitted from the emission layer EML-R, and may thus increase luminous efficiency. Materials which may be included in the hole transport region HTR may be used as materials included in the hole buffer layer. The electron blocking layer is a layer that serves to prevent electrons from being injected from the electron transport region ETR to the hole transport region HTR.

The hole transport region HTR may have a single layer formed of a single material, a single layer formed of a plurality of different materials, or a multilayer structure having a plurality of layers formed of a plurality of different materials. For example, the hole transport region HTR may have a single-layer structure formed of a plurality of different materials, or a structure in which a hole injection layer/hole transport layer, a hole injection layer/hole transport layer/hole buffer layer, a hole injection layer/hole buffer layer, a hole transport layer/hole buffer layer, or a hole injection layer/hole transport layer/electron blocking layer are stacked in order from the first electrode EL1, but the embodiment of the inventive concept is not limited thereto.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The hole injection layer, for example, may include a phthalocyanine compound such as copper phthalocyanine, N,N′-diphenyl-N,N′-bis-[4-(phenyl-m-tolyl-amino)-phenyl]-biphenyl-4,4′-diamine (DNTPD), 4,4′,4″-tris(3-methylphenylphenylamino) triphenylamine (m-MTDATA), 4,4′,4″-tris(N,N-diphenylamino)triphenylamine (TDATA), 4,4′,4″-trisIN,-(2-naphthyl)-N-phenylamino)-triphenylamine (2-TNATA), poly(3,4-ethylenedioxythiophene)/poly(4-styrenesulfonate) (PEDOT/PSS), polyaniline/dodecylbenzenesulfonic acid (PANI/DBS A), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium tetrakis(pentafluorophenyl)borate, dipyrazino[2,3-f: 2′,3′-h]quinoxaline-2,3,6,7,10,11-hexacarbonitrile (HAT-CN), and the like.

For example, the hole transport layer may include carbazole-based derivatives such as N-phenyl carbazole and polyvinyl carbazole, fluorene-based derivatives, N,N′-bis(3-methylphenyl)-N,N′-diphenyl-[1,1-biphenyl]-4,4′-diamine (TPD), triphenylamine-based derivatives such as 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), and the like.

The emission layer EML-R is provided on the hole transport region HTR. The emission layer EML-R may include an organic light emitting material, an inorganic light emitting material, or the like, and may include a micro-scale or nano-scale light emitting body. In the light emitting element ED-1 according to an embodiment, the emission layer EML-R may include a host and a dopant. The light emitting element ED-1 may generate specific light by recombining holes and electrons injected from the first electrode EL1 and the second electrode EL2 in the emission layer EML-R.

In an embodiment, the emission layer EML-R may include quantum dots.

The emission layer EML-R may be formed using various methods such as a vacuum deposition method, a spin coating method, a cast method, a Langmuir-Blodgett (LB) method, an inkjet printing method, a laser printing method, and a laser induced thermal imaging (LITI) method. For example, the emission layer EML-R may be formed by providing an organic light emitting material or an inorganic light emitting material through an inkjet printing method.

The electron transport region ETR is provided on the emission layer EML-R. The electron transport region ETR may include at least one among a hole blocking layer, an electron transport layer, and an electron injection layer, but the embodiment of the inventive concept is not limited thereto.

The electron transport region ETR may have a single layer formed of a single material, a single layer formed of a plurality of different materials, or a multilayer structure having a plurality of layers formed of a plurality of different materials.

For example, the electron transport region ETR may have a single layer structure of an electron injection layer or an electron transport layer, and may have a single layer structure formed of an electron injection material and an electron transport material. In addition, the electron transport region ETR may have a single layer structure formed of a plurality of different materials, or may have a structure in which an electron transport layer/electron injection layer, or a hole blocking layer/electron transport layer/electron injection layer are stacked in order from the emission layer EML-R, but is not limited thereto. The electron transport region ETR may have a thickness of, for example, about 200 Å to about 1500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include an anthracene-based compound. However, the embodiment of the inventive concept is not limited thereto. The electron transport layers may have a thickness of about 100 Å to about 1000 Å, for example, about 150 Å to about 500 Å. When the thicknesses of the electron transport layers satisfy the above-described range, satisfactory electron transport properties may be obtained without a substantial increase in driving voltage.

The electron transport region ETR may include halogenated metals, lanthanide metals, co-deposition materials of a halogenated metal and a lanthanide metal, etc. Meanwhile, the halogenated metal may be an alkali metal halide. For example, the electron transport region ETR may include LiF, lithium quinolate (Liq), Li₂O, BaO, NaCl, CsF, Yb, RbCl, RbI, KI, or KI: Yb, but the embodiment of the inventive concept is not limited thereto. The electron injection layer may also be formed of a mixture material of an electron transport material and an insulating organo-metal salt. For example, the organo-metal salt may include, for example, metal acetates, metal benzoates, metal acetoacetates, metal acetylacetonates, or metal stearates. The electron injection layers may have a thickness of about 1 Å to about 100 Å, for example, about 3 Å to about 90 Å. When the thicknesses of the electron injection layers satisfy the above-described range, satisfactory electron injection properties may be obtained without a substantial increase in driving voltage.

The second electrode EL2 is provided on the electron transport region ETR. The second electrode EL2 may be a common electrode or a cathode. The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound containing thereof (e.g., AgYb, a compound of AgMg and MgYb, etc. depending on the amount) or a mixture containing thereof (e.g., a mixture of Ag and Mg, a mixture of Ag and Yb, etc.). Alternatively, the first electrode EL1 may have a multilayer structure including a reflective film or a transflective film formed of the above-described materials, and a transparent conductive film formed of indium tin oxide (ITO), indium zinc oxide (IZO), zinc oxide (ZnO), indium tin zinc oxide (ITZO), etc.

Although not shown, the second electrode EL2 may be connected with an auxiliary electrode. When the second electrode EL2 is connected with the auxiliary electrode, the resistance of the second electrode EL2 may decrease.

In an embodiment, the light emitting regions PXA-R, PXA-G, and PXA-B may have different areas in size according to the color emitted from the emission layers EML-R, EML-G, and EML-B of the light emitting elements ED-1, ED-2 and ED-3. For example, the third light emitting region PXA-B corresponding to the third light emitting element ED-3 emitting blue light may have a largest area, and the second light emitting region PXA-G corresponding to the second light emitting element ED-2 generating green light may have a smallest area. However, the embodiment of the inventive concept is not limited thereto.

The pixel defining film PDL may separate the light emitting elements ED-1, ED-2 and ED-3. The emission layers EML-R, EML-G, and EML-B of the light emitting elements ED-1, ED-2 and ED-3 may be disposed and separated in opening OH defined by the pixel defining film PDL.

In an embodiment, the pixel defining film PDL may be formed of the photosensitive resin composition described above.

The encapsulation layer TFE may cover the light emitting elements ED-1, ED-2 and ED-3. The encapsulation layer TFE may be a single layer or a laminated layer of a plurality of layers. The encapsulation layer TFE may be a thin film encapsulation layer. The encapsulation layer TFE may be disposed on the light emitting elements ED-1, ED-2, and ED-3 and seal the light emitting elements ED-1, ED-2, and ED-3. The encapsulation layer TFE may cover an upper surface of the second electrode EL2 disposed in the opening OH, and may fill the opening OH. The encapsulation layer TFE may serve to protect the light emitting elements ED-1, ED-2, and ED-3 from moisture/oxygen, and foreign substances such as dust particles.

The encapsulation layer TFE may include at least one organic film or an inorganic film, or may include an organic film and an inorganic film. The encapsulation layer TFE may have a structure in which an organic film and an inorganic film are alternately stacked.

The inorganic film included in the encapsulation layer TFE may include, for example, a silicon nitride layer, a silicon oxy nitride layer, a silicon oxide layer, a titanium oxide layer, or an aluminum oxide layer, and is not particularly limited to the above examples. The organic film included in the encapsulation layer TFE may include an acryl-based organic film, but is not limited to include an acryl-based organic film.

Meanwhile, FIG. 5 shows that the hole transport region HTR and the electron transport region ETR are provided as a continuous common layer covering the pixel defining film PDL. It should be understood that the embodiment is not limited thereto. In an embodiment, the hole transport region HTR and the electron transport region ETR may be disposed in the opening OH defined in the pixel defining film PDL.

For example, when in addition to the emission layers EML-R, EML-G, and EML-B, the hole transport region HTR and the electron transport region ETR are provided through inkjet printing, the hole transport region HTR, the emission layer EML-R, EML-G, and EML-B, and the electron transport region ETR may be provided corresponding to the opening OH defined in the pixel defining film PDL. However, the embodiment is not limited thereto and, regardless of the method of providing each functional layer, as shown in FIG. 5, the hole transport region HTR and the electron transport region ETR are provided as a continuous layer covering the pixel defining film PDL, without being patterned to correspond with openings OH.

Meanwhile, in the display device of an embodiment shown in FIG. 5, although the thicknesses of the emission layers EML-R, EML-G, and EML-B of the first to third light emitting elements ED-1, ED-2, and ED-3 are shown to be similar to one another, the embodiment is not limited thereto. For example, in an embodiment, the thicknesses of the emission layers EML-R, EML-G, and EML-B of the first to third light emitting elements ED-1, ED-2, and ED-3 may be different from one another.

The display module DM according to an embodiment further includes a light control layer PP. In an embodiment, the light control layer PP may include a color filter layer CFL and a planarization layer BL. That is, the display module DM of an embodiment may include the color filter layer CFL disposed on the light emitting elements ED-1, ED-2, and ED-3 of the display panel DP.

The planarization layer BL may be disposed on the color filters CF-R, CF-G, and CF-B to cover the color filters CFR, CF-G, and CF-B. The planarization layer BL may cover irregularities generated in the process of forming the color filters CF-R, CF-G, and CF-B. When needed, a base substrate (not shown) may be disposed on the color filter layer CFL of an embodiment. The base substrate may be a member providing a base surface on which the light control layer PP is disposed. The base substrate may be a glass substrate, a metal substrate, a plastic substrate, and the like. However, the embodiment of the inventive concept is not limited thereto, and the base substrate may be an inorganic layer, an organic layer, or a composite material layer.

In an embodiment, the first filter CF-R may be disposed corresponding to the first light emitting region PXA-R. The second filter CF-G may be disposed corresponding to the second light emitting region PXA-G. The third filter CF-B may be disposed corresponding to the third light emitting region PXA-B.

Referring to FIG. 5, the first filter CF-R may be disposed to overlap the second filter CF-G and the third filter CF-B. In an embodiment, when the first to third light emitting regions PXA-R, PXA-G, and PXA-B are the same in size, the first to third filters CF-R, CF-G, and CF-B may also be the same in size. Meanwhile, in the color filter layer CFL, the first to third filters CF-R, CF-G, and CF-B may be separated by the light blocking unit BM and may not overlap one another. In addition, when the first to third light emitting regions PXA-R, PXA-G, and PXA-B are different in size, the first to third filters CFR, CF-G, and CF-B may be different in height and area, which may thus be different in size. In the display device ES according to an embodiment, the light control layer PP does not include a polarizing layer and may be formed through a low-temperature thermosetting process by including a color filter layer CFL. Accordingly, thermal damage that may occur during the manufacturing process may be prevented.

Hereinafter, embodiments of the inventive concept will be described in more detail through Examples that are set forth to illustrate, but are not to be construed as limiting the present disclosure. Further, unless otherwise specified, “%” and “part” representing the content in the following Examples and Comparative Examples are on a weight basis.

### Example

1. Preparation of Reactive Unsaturated Compound

### Preparation Example 1: Preparation of Compound 1 of Formula 10

Twenty grams of pentaerythritol (Sigma Aldrich) and 42.77 g of acrylic acid (Sigma Aldrich) were put into a three-neck round bottom flask (300 mL) equipped with a distillation tube and a Dean-Stark tube along with 100 g of toluene, and 1 g of sulfuric acid was added thereto, and then the mixture was subjected to a reaction at a raised temperature of 110° C. for 8 hours. Thereafter, at a lowered temperature of 25° C., a reaction solution was washed three times with 200 ml of a 10 wt % Na 2 CO 3 aqueous solution and washed once with 200 ml of water, and an upper organic liquid was dried at 40° C. under reduced pressure to obtain 50 g of Compound 1 of Formula 10. The obtained compound showed a viscosity of 140 mPa·s at 40° C. and a purity of 90% as determined using purity analysis by GC.

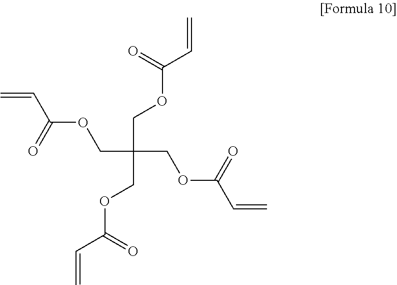 [SMILES: C=CC(=O)OCC(COC(=O)C=C)(COC(=O)C=C)COC(=O)C=C]

### Preparation Example 2: Obtaining Compound 2 Through Purification of Compound 1 of Formula 10

One hundred grams of Silica gel 60 (230-400 mesh, Merck Co.) was filled in a glass column having a diameter of 120 mm, and then 5 g of Compound 1 obtained from Preparation Example 1 was loaded, and hexane and ethyl acetate were mixed in a volume ratio of 4:1, and then 1 L of the mixed solvent was used to perform separation. The obtained Compound 2 (purified product of Compound 1) was dried under reduced pressure at 40° C. to obtain 4.5 g. The obtained Compound 2 showed a viscosity of 170 mPa·s at 40° C. and a purity of 92.7% as determined using purity analysis by gas chromatography (GC).

### Preparation Example 3: Obtaining Compound 3 Through Purification of Compound 1 of Formula 10

Two hundred grams of Silica gel 60 (230-400 mesh, Merck Co.) was filled in a glass column having a diameter of 120 mm, and then 5 g of Compound 1 obtained from Preparation Example 1 was loaded, and hexane and ethyl acetate were mixed in a volume ratio of 6:1, and then 1.5 L of the mixed solvent was used to perform separation. The obtained Compound 3 (purified product of Compound 1) was dried under reduced pressure at 40° C. to obtain 4.2 g. The obtained Compound 3 showed a viscosity of 190 mPa·s at 40° C. and a purity of 93.8% as determined using purity analysis by GC.

### Preparation Example 4: Obtaining Compound 4 Through Purification of Compound 1 of Formula 10

Three hundred grams of Silica gel 60 (230-400 mesh, Merck Co.) was filled in a glass column having a diameter of 120 mm, and then 5 g of Compound 1 obtained from Preparation Example 1 was loaded, and hexane and ethyl acetate were mixed in a volume ratio of 6:1, and then 1.5 L of the mixed solvent was used to perform separation. The obtained Compound 4 (purified product of Compound 1) was dried under reduced pressure at 40° C. to obtain 4.0 g. The obtained Compound 4 showed a viscosity of 196 mPa·s at 40° C. and a purity of 95.8% as determined using purity analysis by GC.

### Preparation Example 5: Obtaining Compound 5 Through Purification of Compound 1 of Formula 10

Four hundred grams of Silica gel 60 (230-400 mesh, Merck Co.) was filled in a glass column having a diameter of 120 mm, and then 5 g of Compound 1 obtained from Preparation Example 1 was loaded and 1 L of hexane was used to perform separation, and hexane and ethyl acetate were mixed in a volume ratio of 6:1, and then 1.5 L of the mixed solvent was used to perform separation. The obtained Compound 5 (purified product of Compound 1) was dried under reduced pressure at 60° C. to obtain 3.6 g. The obtained Compound 5 showed a viscosity of 295 mPa·s at 40° C. and a purity of 99.1% as determined using purity analysis by GC.

### Preparation Example 6: Obtaining Compound 6 Through Purification of Compound 1 of Formula 10

Three hundred and thirty grams of Silica gel 60 (230-400 mesh, Merck Co.) was filled in a glass column having a diameter of 120 mm, and then 5 g of Compound 1 obtained from Preparation Example 1 was loaded and 1 L of hexane was used to perform separation, and hexane and ethyl acetate were mixed in a volume ratio of 6:1, and then 1.5 L of the mixed solvent was used to perform separation. The obtained Compound 6 (purified product of Compound 1) was dried under reduced pressure at 40° C. to obtain 3.6 g. The obtained Compound 6 showed a viscosity of 205 mPa·s at 40° C. and a purity of 98.1% as determined using purity analysis by GC.

### Preparation Example 7: Obtaining Compound 7 Through Purification of Compound 1 of Formula 10

Four hundred grams of Silica gel 60 (230-400 mesh, Merck Co.) was filled in a glass column having a diameter of 120 mm, and then 5 g of Compound 1 obtained from Preparation Example 1 was loaded and 1 L of hexane was used to perform separation, and hexane and ethyl acetate were mixed in a volume ratio of 6:1, and then 1.5 L of the mixed solvent was used to perform separation. The obtained Compound 7 (purified product of Compound 1) was dried under reduced pressure at 40° C. to obtain 3.6 g. The obtained Compound 7 showed a viscosity of 230 mPa·s at 40° C. and a purity of 98.8% as determined using purity analysis by GC.

### Preparation Example 8: Obtaining Compound 8 Through Purification of Compound 1 of Formula 10

Four hundred and twenty grams of Silica gel 60 (230-400 mesh, Merck Co.) was filled in a glass column having a diameter of 120 mm, and then 5 g of Compound 1 obtained from Preparation Example 1 was loaded and 1 L of hexane was used to perform separation, and hexane and ethyl acetate were mixed in a volume ratio of 6:1, and then 1.5 L of the mixed solvent was used to perform separation. The obtained Compound 8 (purified product of Compound 1) was dried under reduced pressure at 40° C. to obtain 3.6 g. The obtained Compound 8 showed a viscosity of 250 mPa·s at 40° C. and a purity of 99.2% as determined using purity analysis by GC.

### Preparation Example 9: Obtaining Compound 9 Through Purification of Compound 1 of Formula 10

Four hundred and fifty grams of Silica gel 60 (230-400 mesh, Merck Co.) was filled in a glass column having a diameter of 120 mm, and then 5 g of Compound 1 obtained from Preparation Example 1 was loaded and 1 L of hexane was used to perform separation, and hexane and ethyl acetate were mixed in a volume ratio of 6:1, and then 1.5 L of the mixed solvent was used to perform separation. The obtained Compound 9 (purified product of Compound 1) was dried under reduced pressure at 40° C. to obtain 3.6 g. The obtained Compound 9 showed a viscosity of 275 mPa·s at 40° C. and a purity of 99.6% as determined using purity analysis by GC.

### Preparation Example 10: Obtaining Compound 10 through purification of Compound 1 of Formula 10

Three hundred and fifteen grams of Silica gel 60 (230-400 mesh, Merck Co.) was filled in a glass column having a diameter of 120 mm, and then 5 g of Compound 1 obtained from Preparation Example 1 was loaded and 1 L of hexane was used to perform separation, and hexane and ethyl acetate were mixed in a volume ratio of 6:1, and then 1.5 L of the mixed solvent was used to perform separation. The obtained Compound 10 (purified product of Compound 1) was dried under reduced pressure at 40° C. to obtain 3.5 g. The obtained Compound 10 showed a viscosity of 200 mPa·s at 40° C. and a purity of 97.9% as determined using purity analysis by GC.

### Preparation Example 11: Obtaining Compound 11 Through Purification of Compound 1 of Formula 10

Three hundred and sixty grams of Silica gel 60 (230-400 mesh, Merck Co.) was filled in a glass column having a diameter of 120 mm, and then 5 g of Compound 1 obtained from Preparation Example 1 was loaded and 1 L of hexane was used to perform separation, and hexane and ethyl acetate were mixed in a volume ratio of 6:1, and then 1.5 L of the mixed solvent was used to perform separation. The obtained Compound 11 (purified product of Compound 1) was dried under reduced pressure at 40° C. to obtain 3.5 g. The obtained Compound 11 showed a viscosity of 215 mPa·s at 40° C. and a purity of 98.5% as determined using purity analysis by GC.

### Preparation Example 12: Obtaining Compound 12 Through Purification of Compound 1 of Formula 10

Four hundred seventy grams of Silica gel 60 (230-400 mesh, Merck Co.) was filled in a glass column having a diameter of 120 mm, and then 5 g of Compound 1 obtained from Preparation Example 1 was loaded and 1 L of hexane was used to perform separation, and hexane and ethyl acetate were mixed in a volume ratio of 6:1, and then 1.5 L of the mixed solvent was used to perform separation. The obtained Compound 12 (purified product of Compound 1) was dried under reduced pressure at 40° C. to obtain 3.2 g. The obtained Compound 12 showed a viscosity of 280 mPa·s at 40° C. and a purity of 99.5% as determined using purity analysis by GC.

2. Preparation of Other Materials

### Preparation Example 13: Preparation of Compound 13

In a flask (300 mL) equipped with a cooling tube and a stirrer, 34.2 g of styrene (Aldrich), 10.8 g of methacrylic acid (Aldrich), 9 g of phenylmaleimide (TCI), 6 g of methyl methacrylate (Aldrich), and 6 g of 2,2′-azobis(2,4-dimethylvaleronitrile) (initiator) were added together with propylene glycol monomethyl ether acetate, and the mixture was stirred at a raised temperature of 80° C. for 4 hours to obtain Compound 10 having a weight average molecular weight of 11,000 as a solution having a solid content of 30%.

Preparation Example 14: Preparation of Black Pigment Dispersion

Fifteen grams of black pigment (Irgaphor Black S0100 CF, BASF), 8.5 g of dispersant (disperbyk 163, BYK), 7.5 g of Compound 13, 69 g of propylene glycol methyl ether acetate, and 100 g of zirconia beads having a diameter of 0.5 mm (Toray) were dispersed for 10 hours using a paint shaker (Asada) to obtain a black pigment dispersion.

3. Preparation of Photosensitive Resin Composition

The photosensitive resin compositions of Examples 1 to 7 and Comparative Examples 1 to 7 were prepared according to the compositions shown in Tables 1 and 2 below (unit: wt %). In Tables 1 and 2 below, Examples 1 to 7 are compositions satisfying the viscosity range of a reactive unsaturated compound included in a photosensitive resin composition according to an embodiment of the inventive concept, and Comparative Examples 1 to 7 are compositions having less than 3 ethylenically unsaturated double bond groups or out of the viscosity range of a reactive unsaturated compound included in a photosensitive resin composition according to an embodiment of the inventive concept.

TABLE 1

| Item | | Example 1 | Example 2 | Example 3 | Example 4 | Example 5 | Example 6 | Example 7 |
|---|---|---|---|---|---|---|---|---|
| Pigment | Black pigment | 30 | 30 | 30 | 30 | 30 | 30 | 30 |
| | dispersion of | | | | | | | |
| | Preparation | | | | | | | |
| | Example 14 | | | | | | | |
| Alkali | V259ME | 9.2 | 9.2 | 9.2 | 9.2 | 9.2 | 9.2 | 9.2 |
| soluble | (NIPPON STEEL | | | | | | | |
| resin | CHEMICAL) | | | | | | | |
| Initiator | OXE-02 (BASF) | 0.8 | 0.8 | 0.8 | 0.8 | 0.8 | 0.8 | 0.8 |
| Reactive | Compound 1 | | | | | | | |
| unsaturated | of Preparation | | | | | | | |
| compound | Example 1 | | | | | | | |
| Reactive | Compound 2 | | | | | | | |
| unsaturated | of Preparation | | | | | | | |
| compound | Example 2 | | | | | | | |
| Reactive | Compound 3 | | | | | | | |
| unsaturated | of Preparation | | | | | | | |
| compound | Example 3 | | | | | | | |
| Reactive | Compound 4 | | | | | | | |
| unsaturated | of Preparation | | | | | | | |
| compound | Example 4 | | | | | | | |
| Reactive | Compound 5 | | | | | | | |
| unsaturated | of Preparation | | | | | | | |
| compound | Example 5 | | | | | | | |
| Reactive | Compound 6 | 5 | | | | | | |
| unsaturated | of Preparation | | | | | | | |
| compound | Example 6 | | | | | | | |
| Reactive | Compound 7 | | 5 | | | | | |
| unsaturated | of Preparation | | | | | | | |
| compound | Example 7 | | | | | | | |
| Reactive | Compound 8 | | | 5 | | | | |
| unsaturated | of Preparation | | | | | | | |
| compound | Example 8 | | | | | | | |
| Reactive | Compound 9 | | | | 5 | | | |
| unsaturated | of Preparation | | | | | | | |
| compound | Example 9 | | | | | | | |
| Reactive | Compound 10 | | | | | 5 | | |
| unsaturated | of Preparation | | | | | | | |
| compound | Example 10 | | | | | | | |
| Reactive | Compound 11 | | | | | | 5 | |
| unsaturated | of Preparation | | | | | | | |
| compound | Example 11 | | | | | | | |
| Reactive | Compound 12 | | | | | | | 5 |
| unsaturated | of Preparation | | | | | | | |
| compound | Example 12 | | | | | | | |
| Reactive | M200 | | | | | | | |
| unsaturated | (bifunctional | | | | | | | |
| compound | monomer, Miwon | | | | | | | |
| | Commercial | | | | | | | |
| | Co., Ltd.) | | | | | | | |
| Reactive | n-Butyl Acetate | | | | | | | |
| unsaturated | (monofunctional | | | | | | | |
| compound | monomer, Sigma | | | | | | | |
| | Aldrich) | | | | | | | |
| Solvent | Propylene glycol | 55 | 55 | 55 | 55 | 55 | 55 | 55 |
| | methyl ether acetate | | | | | | | |

TABLE 2

| | | Comparative | Comparative | Comparative | Comparative | Comparative | Comparative | Comparative |
|---|---|---|---|---|---|---|---|---|
| Item | | Example 1 | Example 2 | Example 3 | Example 4 | Example 5 | Example 6 | Example 7 |
| Pigment | Black pigment | 30 | 30 | 30 | 30 | 30 | 30 | 30 |
| | dispersion of | | | | | | | |
| | Preparation | | | | | | | |
| | Example 14 | | | | | | | |
| Alkali | V259ME | 9.2 | 9.2 | 9.2 | 9.2 | 9.2 | 9.2 | 9.2 |
| soluble | (NIPPON STEEL | | | | | | | |
| resin | CHEMICAL) | | | | | | | |
| Initiator | OXE-02 (BASF) | 0.8 | 0.8 | 0.8 | 0.8 | 0.8 | 0.8 | 0.8 |
| Reactive | Compound 1 | 5 | | | | | | |
| unsaturated | of Preparation | | | | | | | |
| compound | Example 1 | | | | | | | |
| Reactive | Compound 2 | | 5 | | | | | |
| unsaturated | of Preparation | | | | | | | |
| compound | Example 2 | | | | | | | |
| Reactive | Compound 3 | | | 5 | | | | |
| unsaturated | of Preparation | | | | | | | |
| compound | Example 3 | | | | | | | |
| Reactive | Compound 4 | | | | 5 | | | |
| unsaturated | of Preparation | | | | | | | |
| compound | Example 4 | | | | | | | |
| Reactive | Compound 5 | | | | | 5 | | |
| unsaturated | of Preparation | | | | | | | |
| compound | Example 5 | | | | | | | |
| Reactive | Compound 6 | | | | | | | |
| unsaturated | of Preparation | | | | | | | |
| compound | Example 6 | | | | | | | |
| Reactive | Compound 7 | | | | | | | |
| unsaturated | of Preparation | | | | | | | |
| compound | Example 7 | | | | | | | |
| Reactive | Compound 8 | | | | | | | |
| unsaturated | of Preparation | | | | | | | |
| compound | Example 8 | | | | | | | |
| Reactive | Compound 9 | | | | | | | |
| unsaturated | of Preparation | | | | | | | |
| compound | Example 9 | | | | | | | |
| Reactive | Compound 10 | | | | | | | |
| unsaturated | of Preparation | | | | | | | |
| compound | Example 10 | | | | | | | |
| Reactive | Compound 11 | | | | | | | |
| unsaturated | of Preparation | | | | | | | |
| compound | Example 11 | | | | | | | |
| Reactive | Compound 12 | | | | | | | |
| unsaturated | of Preparation | | | | | | | |
| compound | Example 12 | | | | | | | |
| Reactive | M200 | | | | | | 5 | |
| unsaturated | (bifunctional | | | | | | | |
| compound | monomer, Miwon | | | | | | | |
| | Commercial | | | | | | | |
| | Co., Ltd.) | | | | | | | |
| Reactive | n-Butyl Acetate | | | | | | | 5 |
| unsaturated | (monofunctional | | | | | | | |
| compound | monomer, Sigma | | | | | | | |
| | Aldrich) | | | | | | | |
| Solvent | Propylene glycol | 55 | 55 | 55 | 55 | 55 | 55 | 55 |
| | methyl ether acetate | | | | | | | |

4. Preparation and Evaluation of Pixel Defining Film

(1) Preparation of Pixel Defining Film

1) Forming Coating Films

The photosensitive resin compositions according to Examples 1 to 7 and Comparative Examples 1 to 7 were applied onto a washed 10 cm×10 cm glass substrate to a thickness of 1 μm using a spin coater, and then heated at 100° C. for 1 minute to remove a solvent, thereby forming coating films.

2) Exposure

Masks were placed on the obtained coating films, which were then irradiated with actinic rays of 190 nm to 500 nm to form exposure patterns. An exposure machine MA-6 (SUSS) was used and an exposure amount of 100 mJ/cm 2 was applied for irradiation.

3) Development

After the exposure, the exposed patterns were developed by dipping the patterns in an AX300 MIF developer (AZEM) at 25° C. for 1 minute, and washed with water to dissolve and remove an unexposed portion, leaving only an exposed portion to form image patterns.

4) Post-Processing

The image patterns obtained through the above development was post-baked in a 230° C. oven for 30 minutes.

(2) Evaluation of Resolution and Adhesion

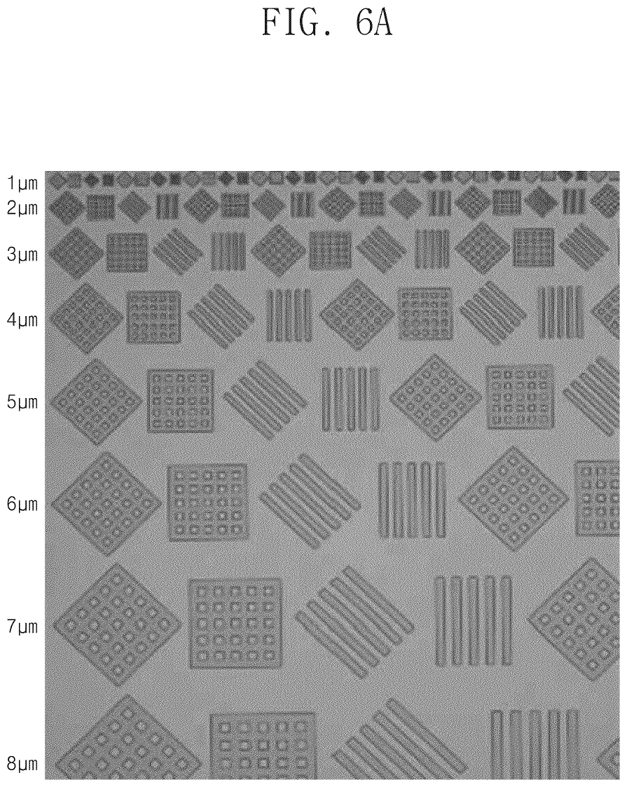

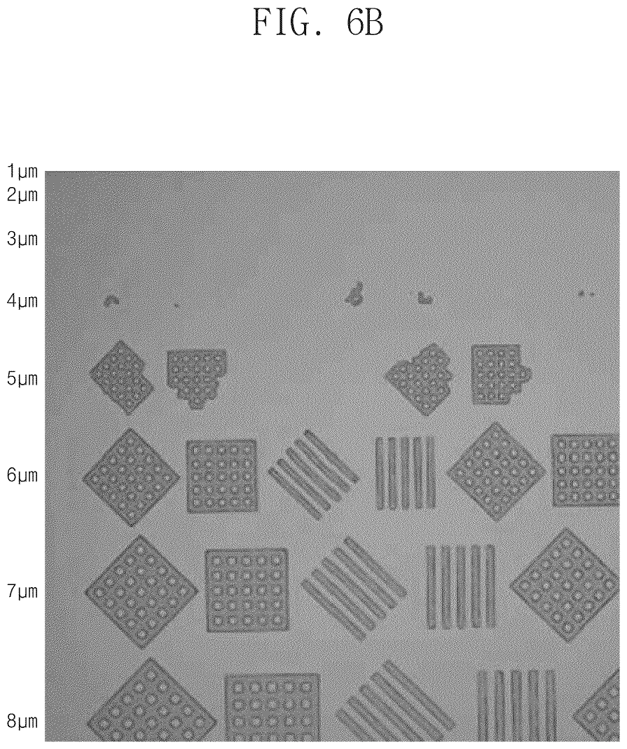

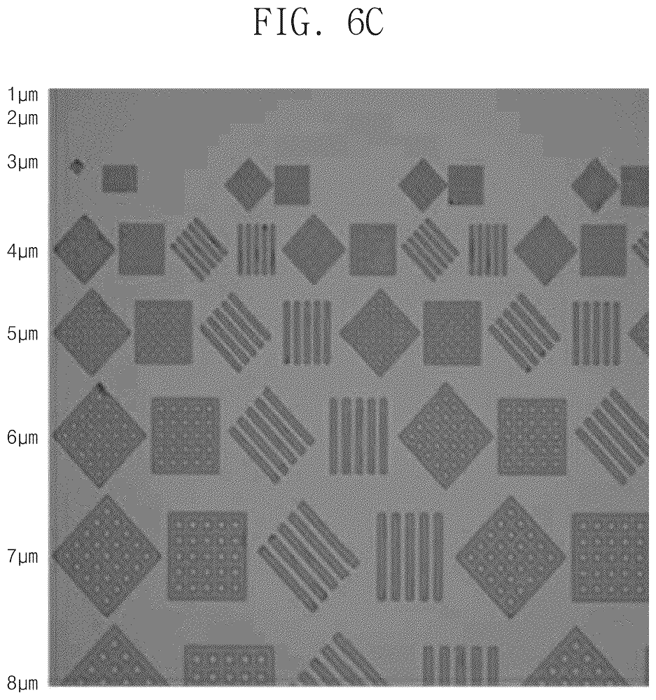

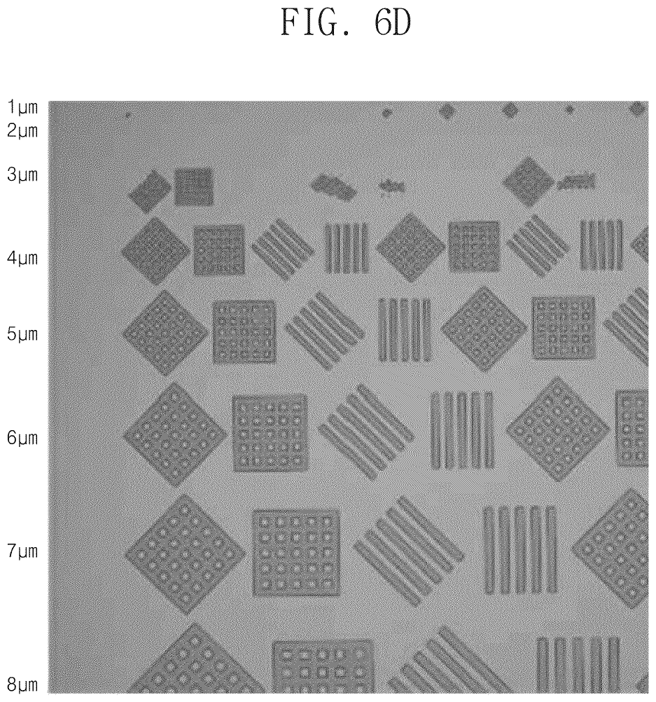

Pattern quality was evaluated using an optical microscope (Nikon Co.) for the patterns manufactured through the method of preparing a pixel defining film described above. When observed with the optical microscope, a minimum size of the patterns attached to a substrate without breakup or residue was photographed and shown in FIGS. 6A to 6D, and the results are summarized in Table 3 below. FIG. 6A is an image showing results of evaluation on resolution and adhesion of Example 1, FIG. 6B is an image showing results of evaluation on resolution and adhesion of Comparative Example 1, FIG. 6C is an image showing results of evaluation on resolution and adhesion of Comparative Example 2, and FIG. 6D is an image showing results of evaluation on resolution and adhesion of Comparative Example 3.

TABLE 3

| | | | | | | | | Compar- |
|---|---|---|---|---|---|---|---|---|
| | | | | | | | | ative |
| | Exam- | Exam- | Exam- | Exam- | Exam- | Exam- | Exam- | Exam- |
| | ple 1 | ple 2 | ple 3 | ple 4 | ple 5 | ple 6 | ple 7 | ple 1 |
| Size of pattern | 1 | 1 | 1 | 1 | 1 | 1 | 1 | 8 |
| with minimum | | | | | | | | |
| size on substrate | | | | | | | | |
| (μm) | | | | | | | | |

| | Compar- | Compar- | Compar- | Compar- | Compar- | Compar- |
|---|---|---|---|---|---|---|
| | ative | ative | ative | ative | ative | ative |
| | Exam- | Exam- | Exam- | Exam- | Exam- | Exam- |
| | ple 2 | ple 3 | ple 4 | ple 5 | ple 6 | ple 7 |
| Size of pattern | 6 | 4 | 3 | 6 | 22 | — |
| with minimum | | | | | | |
| size on substrate | | | | | | |
| (μm) | | | | | | |

Referring to Table 3 and FIG. 6A, it is seen that when patterns were formed using the photosensitive resin compositions of Examples 1 to 7, adhesion was excellent, and even when patterns were formed in a size of 1 μm, breakup or residue of the patterns was not caused and high-resolution pattern properties were observed.

However, it is seen that when the photosensitive resin composition of Comparative Example 1 was used, fine patterns having a size of 1 μm to 4 μm were hardly formed, and even when patterns were formed to have a size of 5 μm or greater, breakup or residue of some patterns was observed (see FIG. 6B). In addition, it is seen that even when the photosensitive resin compositions of Comparative Examples 2 to 4 were used, pattern properties were remarkably degraded compared to Examples 1 to 4. Comparative Example 5, in which drying was performed at a relatively high temperature and some polymerization took place, had a relatively high viscosity of 295 mPa·s, and showed high purity but low resolution upon patterning as GC was not able to confirm polymerized materials. In addition, when using M200 (manufactured by Miwon Commercial Co., Ltd.) having two ethylenically unsaturated double bond groups as in Comparative Example 6, degree of cure was low, resulting in fairly low resolution. As in Comparative Example 7, when n-Butyl methacrylate having one ethylenically unsaturated double bond group was applied, it is seen that all patterns in a substrate were washed out because curing was performed well.

A photosensitive resin composition of an embodiment includes a reactive unsaturated compound having at least three ethylenically unsaturated double bond groups and exhibiting viscosity properties within a specific numerical range, thereby improving adhesion to a substrate with high sensitivity. Accordingly, pattern breakup or residue upon photolithography may be prevented to form patterns with high resolution.

A display device of an embodiment includes a pixel defining film formed using the photosensitive resin composition described above, and may thus provide excellent color and reduce reflection of external light.

A photosensitive resin composition of an embodiment includes a specific reactive unsaturated compound, and may thus improve adhesion to a substrate with high sensitivity. Accordingly, pattern breakup or residue upon photolithography may be prevented to form patterns with high resolution. In addition, the photosensitive resin composition of an embodiment may prevent patterns from being inversely tapered after a developing process of photolithography.

A display device of an embodiment includes a pixel defining film formed using the photosensitive resin composition described above, and may thus exhibit excellent color, reduced external light reflectance, and improved reliability.

Although the present disclosure has been described with reference to a preferred embodiment of the inventive concept, it will be understood that the inventive concept should not be limited to these preferred embodiments but various changes and modifications can be made by those skilled in the art without departing from the spirit and scope of the present disclosure.

Hence, the technical scope of the present disclosure is not limited to the detailed descriptions in the specification but should be determined only with reference to the claims.

What is claimed is:

1. A photosensitive resin composition comprising:
a reactive unsaturated compound that includes an acryl-based compound having at least three ethylenically unsaturated double bond groups in molecules and having a viscosity of about 200 mPa·s to about 280 mPa·s at 40° C.;
an alkali soluble resin;
an initiator; and
a pigment.

2. The photosensitive resin composition of claim 1, wherein the acryl-based compound is represented by Formula 1 below: 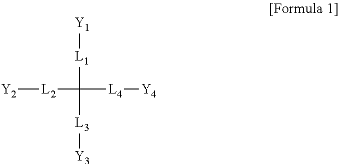
wherein in Formula 1 above,
L₁ to L₄ are each independently a substituted or unsubstituted alkylene group having 1 to 25 carbon atoms,
Y₁ to Y₄ are each independently a hydrogen atom, a hydroxy group or a substituted or unsubstituted acrylic acid derivative, and
at least three of Y₁ to Y₄ include a substituted or unsubstituted acrylic acid derivative.

3. The photosensitive resin composition of claim 2, wherein the acryl-based compound represented by Formula 1 above is represented by one of Formula 2 and Formula 3 below: 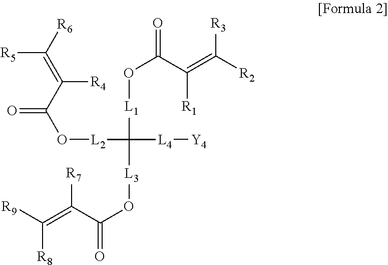
wherein in Formula 2 above, L₁ to L₄ are the same as defined in Formula 1 above,
R₁ to R₉ are each independently a hydrogen atom, a deuterium atom, a substituted or unsubstituted alkyl group having 1 to 20 carbon atoms, or a substituted or unsubstituted aryl group having 6 to 24 ring-forming carbon atoms, and
Y₄ is a hydrogen atom or a hydroxy group, and 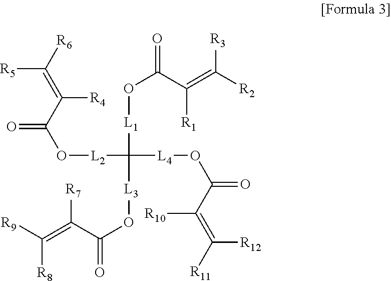
wherein in Formula 3 above, L₁ to L₄ are the same as defined in Formula 1 above, and
R₁ to R₁₂ are each independently a hydrogen atom, a deuterium atom, a substituted or unsubstituted alkyl group having 1 to 20 carbon atoms, or a substituted or unsubstituted aryl group having 6 to 24 ring-forming carbon atoms.

4. The photosensitive resin composition of claim 1, wherein the reactive unsaturated compound further comprises at least one of a monomer or an oligomer, which has at least one ethylenically unsaturated double bond.

5. The photosensitive resin composition of claim 1, wherein the alkali soluble resin comprises at least one of a cardo-based resin, an acryl-based resin, a polyester-based resin, a polyurethane-based resin, a polysiloxane resin, a polycyclic side chain-containing resin, or an acid-modified epoxy resin.

6. The photosensitive resin composition of claim 5, wherein the cardo-based resin has a weight average molecular weight of about 1,000 g/mol to about 100,000 g/mol.

7. The photosensitive resin composition of claim 1, wherein the pigment comprises at least one of a black organic pigment, a black inorganic pigment, or a mixture of two or more coloring pigments.

8. The photosensitive resin composition of claim 1, further comprising a solvent.

9. The photosensitive resin composition of claim 8, wherein with respect to a total weight of a composition, the photosensitive resin composition comprises:
the acryl-based compound in an amount of about 0.3 wt % to about 30 wt %;
the alkali soluble resin in an amount of about 1 wt % to about 30 wt %;
the initiator in an amount of about 0.01 wt % to about 10 wt %;
the pigment in an amount of about 1 wt % to about 30 wt %; and
the solvent in a residual amount.

10. The photosensitive resin composition of claim 7, wherein the black organic pigment comprises at least one of a benzofuranone-based black pigment, a perylene-based black pigment, an azo-based black pigment, lactam black, aniline black, or an indolinone-based black pigment.

11. The photosensitive resin composition of claim 7, wherein the black organic pigment comprises a coating layer on a surface,
the coating layer including at least one of a silica coating layer, a metal oxide coating layer, or a metal hydroxide coating layer.

12. A display device comprising:
a display panel including a plurality of light emitting elements and a pixel defining film separating the plurality of light emitting elements; and
a light control layer disposed on the display panel,
wherein the pixel defining film is formed of a photosensitive resin composition including a reactive unsaturated compound containing an acryl-based compound having at least three ethylenically unsaturated double bond groups in molecules and having a viscosity of about 200 mPa s to about 280 mPa s at 40° C.; an alkali soluble resin; an initiator; and a pigment.

13. The display device of claim 12, wherein the acryl-based compound is represented by Formula 1 below: 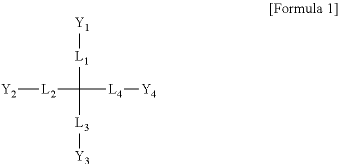
wherein in Formula 1 above,
L₁ to L₄ are each independently a substituted or unsubstituted alkylene group having 1 to 25 carbon atoms,
Y₁ to Y₄ are each independently a hydroxy group or a substituted or unsubstituted acrylic acid derivative, and
at least three of Y₁ to Y₄ include a substituted or unsubstituted acrylic acid derivative.

14. The display device of claim 13, wherein the acryl-based compound represented by Formula 1 above is represented by Formula 2 or Formula 3 below: 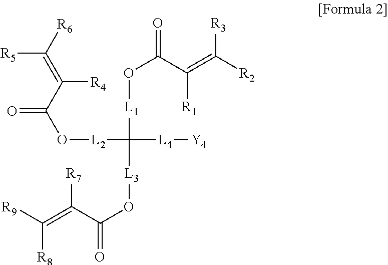
wherein in Formula 2 above, L₁ to L₄ are the same as defined in Formula 1 above, R₁ to R₉ are each independently a hydrogen atom, a deuterium atom, a substituted or unsubstituted alkyl group having 1 to 20 carbon atoms, or a substituted or unsubstituted aryl group having 6 to 24 carbon atoms, and
Y₄ is a hydrogen atom or a hydroxy group, and 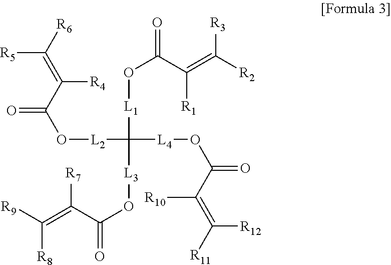
wherein in Formula 3 above, L₁ to L₄ are the same as defined in Formula 1 above, and
R₁ to R₁₂ are each independently a hydrogen atom, a deuterium atom, a substituted or unsubstituted alkyl group having 1 to 20 carbon atoms, or a substituted or unsubstituted aryl group having 6 to 24 carbon atoms.

15. The display device of claim 12, wherein the alkali soluble resin comprises at least one of a cardo-based resin, an acryl-based resin, a polyester-based resin, a polyurethane-based resin, a polysiloxane resin, a polycyclic side chain-containing resin, or an acid-modified epoxy resin.

16. The display device of claim 15, wherein the cardo-based resin has a weight average molecular weight of about 1,000 g/mol to about 100,000 g/mol.

17. The display device of claim 12, wherein the pigment comprises at least one of a black organic pigment, a black inorganic pigment, or a mixture of two or more coloring pigments.

18. The display device of claim 12, wherein the photosensitive resin composition further comprises a solvent.

19. The display device of claim 18, wherein with respect to a total weight of a composition, the photosensitive resin composition comprises:
the acryl-based compound in an amount of about 0.3 wt % to about 30 wt %;
the alkali soluble resin in an amount of about 1 wt % to about 30 wt %;
the photopolymerization initiator in an amount of about 0.01 wt % to about 10 wt %;
the pigment in an amount of about 1 wt % to about 30 wt %; and
the solvent in a residual amount.

20. The display device of claim 17, wherein the black organic pigment comprises at least one of a benzofuranone-based black pigment, a perylene-based black pigment, an azo-based black pigment, lactam black, aniline black, or an indolinone-based black pigment.

21. The display device of claim 17, wherein the black organic pigment comprises a coating layer on a surface,
the coating layer including at least one of a silica coating layer, a metal oxide coating layer, or a metal hydroxide coating layer.
